(19) United States
(12) Patent Application Publication
(10) Pub. No.: US 2008/0279776 A1
(43) Pub. Date: Nov. 13, 2008

(54) Photosensitizers and MRI Enhancers

(75) Inventors: William H. Ayer Porter, Midelton (IE); Margaret Ayer Porter, Midelton (IE); Alexander Ovchinnikov, Midelton (IE)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(73) Assignee: Photo Diagnostic Devices (PDD) Limited

(21) Appl. No.: 11/910,857

(22) PCT Filed: Apr. 7, 2005
(86) PCT No.: PCT/IB2005/051142

§ 371 Date: Oct. 16, 2007

(51) Int. Cl. A61K 49/06; A61K 31/555; A61K 49/00; A61B 5/055; C07D 209/56; A61L 2/18

(52) U.S. Cl. 424/9.3; 514/185; 424/9.1; 548/402; 422/28

(57) ABSTRACT

The present invention relates to the use of a compound of formula 3 or a salt thereof for the manufacture of a medicament or phototherapeutic agent for the treatment of acne, Aids, viral hepatitis, diabetic retinopathy, infection with sars virus, coronary artery stenosis, carotid artery stenosis, intermittent claudication, Asian (chicken) flu virus, cervical dysplasia or cancer of the blood, cervix, naso-pharynx, trachea, larynx, bronchi, bronchioles, bladder, esophagus, stomach, rectum, colon, prostate, hollow organs, bile duct, ureter, kidney, uterus, vaginal or other female adnexa. The invention also relates to methods of treating these diseases.

The present invention further relates to the use of a compound of formula 3 or a salt thereof for the manufacture of a photodiagnostic agent for the detection of the above diseases, as well as atherosclerosis, multiple sclerosis, diabetes, arthritis, rheumatoid arthritis, a fungal, viral, chlamydial, bacterial, nanobacterial or parasitic infectious disease, HIV, hepatitis, herpes simplex, herpes zoster, psoriasis, a cardiovascular disease, or a dermatological condition. The invention also relates to methods of detecting these diseases by photodiagnosis.

The present invention further relates to a method of cold sterilising a surgical or other device, comprising the steps of: providing a compound of formula 3 or a salt thereof on the device and subjecting the device to irradiation or sound.

The present invention further relates to a compound of formula 3 or a salt thereof, linked or attached to a magnetic element. Such a compound may be used as an MRI enhancer. The present invention also relates to a method of carrying out an MRI scan using such an MRI enhancer.

### TECHNICAL FIELD

The present invention relates to the use of a compound of formula 3 or a salt thereof for the manufacture of a medicament or phototherapeutic agent for the treatment of acne, Aids, viral hepatitis, diabetic retinopathy, infection with sars virus, coronary artery stenosis, carotid artery stenosis, intermittent claudication, Asian (chicken) flu virus, cervical dysplasia or cancer of the blood, cervix, naso-pharynx, trachea, larynx, bronchi, bronchioles, bladder, esophagus, stomach, rectum, colon, prostate, hollow organs, bile duct, ureter, kidney, uterus, vaginal or other female adnexa. The invention also relates to methods of treating these diseases.

The present invention further relates to the use of a compound of formula 3 or a salt thereof for the manufacture of a photodiagnostic agent for the detection of the above diseases, as well as atherosclerosis, multiple sclerosis, diabetes, arthritis, rheumatoid arthritis, a fungal, viral, chlamydial, bacterial, nanobacterial or parasitic infectious disease, HIV, hepatitis, herpes simplex, herpes zoster, psoriasis, a cardiovascular disease, or a dermatological condition. The invention also relates to methods of detecting these diseases by photodiagnosis.

The present invention further relates to a method of cold sterilising a surgical or other device, comprising the steps of: providing a compound of formula 3 or a salt thereof on the device and subjecting the device to irradiation or sound.

The present invention further relates to a compound of formula 3 or a salt thereof, linked or attached to a magnetic element. Such a compound may be used as an MRI enhancer. The present invention also relates to a method of carrying out an MRI scan using such an MRI enhancer.

### BACKGROUND ART

Photodynamic therapy (PDT) is a known treatment that uses light to destroy, for example, cancer tissue. Cytoluminescent therapy (CLT) is a form of photodynamic therapy. In both photodynamic therapy and cytoluminescent therapy, a photosensitizer is administered to a patient, generally orally or intravenously. The photosensitizer collects selectively in, for example, cancer tissue, other diseased cells, cholesterol plaques, new vessels, viruses, bacteria and fungi. When exposed to light, the photosensitizer becomes activated, releasing a highly energized, free radical form of oxygen known as singlet oxygen. Singlet oxygen destroys the cancer tissue, other diseased cells etc. from the inside out, while leaving normal tissues largely unaffected. The administered photosensitizer can be exposed to light and activated internally using fibre-optic catheters or endoscopes inserted into the body to bring the light directly to the seat of the cancer tissue, other diseased cells etc., or externally using light of higher wavelengths, which allows a greater depth of penetration into the body.

Most known photosensitizers have mayor drawbacks, for example, they may be difficult to prepare and purify, or they may only accumulate slowly in tumours. For example, Russian patent RU-2183956 discloses photosensitizers based on a mixture of alkali metal salts, chlorine-e6, purpurine-5 and purpurine-18, which is obtained by extracting Spirulina biomass. However, the photosensitizers disclosed in RU-2183956 have a low selectivity for tumour tissues, a high toxicity to normal organs and tissues, and a low therapeutic photoactivity in tumour cells. Moreover, they are chemically and photochemically unstable, but are only slowly metabolised and cleared from normal tissues.

It is therefore an object of the present invention to provide novel uses of photosensitizers with certain desired physical, chemical, photophysical and biological properties, such as high selectivity for tumour or other diseased tissue, optimum speed of accumulation in tumour or other diseased tissue, rapid clearance from normal tissue, slow clearance from tumour or other diseased tissue, high photodynamic activity, low tendency to induce photosensitivity, low cytotoxicity towards normal tissue, homogeneity and chemical stability of medicinal forms during storage, and ease of preparation and purification of industrial quantities.

The inventors of the present invention have investigated the compound of formula 1, 18-carboxy-20-(carboxymethyl)-8-ethenyl-13-ethyl-2,3-dihydro-3,7,12,17-tetramethyl-21H,23H-porphine-2-propanoic acid, which is also known as phytochlorin or chlorine-e6, and derivatives and metal complexes thereof.

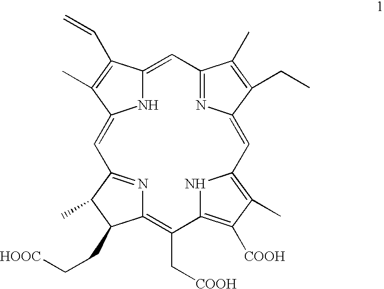

The inventors of the present invention have further developed a process for the preparation of derivatives and metal complexes of chlorine-e6, which is simple and effective, and provides the derivatives and metal complexes without residual toxic reagents. This process is disclosed in co-owned international patent application no. PCT/IB2004/051998, which is hereby incorporated by reference in its entirety, in particular, in respect of the process for the preparation of derivatives and metal complexes of chlorine-e6 described therein.

### SUMMARY OF THE INVENTION

A first aspect of the present invention is the use of a compound for the manufacture of a medicament for the treatment of acne, Aids, viral hepatitis, diabetic retinopathy, infection with sars virus, coronary artery stenosis, carotid artery stenosis, intermittent claudication, Asian (chicken) flu virus, cervical dysplasia or cancer of the blood, cervix, naso-pharynx, trachea, larynx, bronchi, bronchioles, bladder, esophagus, stomach, rectum, colon, prostate, hollow organs, bile duct, ureter, kidney, uterus, vaginal or other female adnexa, wherein the compound is of formula 3

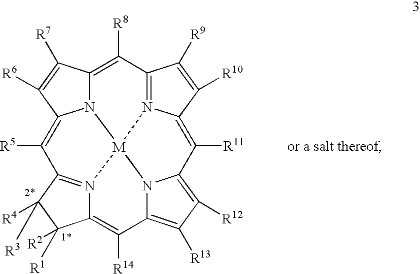

wherein
M is a metal atom in the M(II) oxidation state, a metal halide, a metal oxide or a silicon with two substituents,
each R¹, R², R³, R⁴, R⁵, R⁶, R⁷, R⁸, R⁹, R¹⁰, R¹¹, R¹², R¹³ and R¹⁴ is independently hydrogen, (CH₂)ₙ—CHO, (CH₂)ₙ—CO₂R¹⁵ or a C₁-C₆ saturated or unsaturated alkyl group optionally substituted with one or more of —OH and —NH₂,
n is 0, 1, 2 or 3, and
each R¹⁵ is independently hydrogen, lithium, sodium, potassium, magnesium, calcium, a C₁-C₆ saturated or unsaturated alkyl group optionally substituted with one or more of —OH and —NH₂, or a naturally occurring amino acid.

Preferably the medicament is a phototherapeutic agent for the use in photodynamic therapy or cytoluminescent therapy for the treatment of acne, Aids, viral hepatitis, diabetic retinopathy, infection with sars virus, coronary artery stenosis, carotid artery stenosis, intermittent claudication, Asian (chicken) flu virus, cervical dysplasia or cancer of the blood, cervix, naso-pharynx, trachea, larynx, bronchi, bronchioles, bladder, esophagus, stomach, rectum, colon, prostate, hollow organs, bile duct, ureter, kidney, uterus, vaginal or other female adnexa.

The first aspect of the present invention further provides the use of a compound for the manufacture of a photodiagnostic agent for the detection of acne, Aids, viral hepatitis, diabetic retinopathy, infection with sars virus, coronary artery stenosis, carotid artery stenosis, intermittent claudication, Asian (chicken) flu virus, cervical dysplasia, or cancer of the blood, cervix, naso-pharynx, trachea, larynx, bronchi, bronchioles, bladder, esophagus, stomach, rectum, colon, prostate, hollow organs, bile duct, ureter, kidney, uterus, vaginal or other female adnexa, or atherosclerosis, multiple sclerosis, diabetes, arthritis, rheumatoid arthritis, a fungal, viral, chlamydial, bacterial, nanobacterial or parasitic infectious disease, HIV, hepatitis, herpes simplex, herpes zoster, psoriasis, a cardiovascular disease, or a dermatological condition, wherein the compound is of formula 3

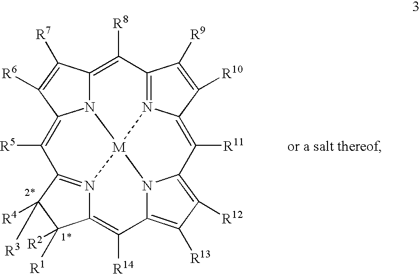

wherein
M is a metal atom in the M(II) oxidation state, a metal halide, a metal oxide or a silicon with two substituents,
each R¹, R², R³, R⁴, R⁵, R⁶, R⁷, R⁸, R⁹, R¹⁰, R¹¹, R², R³ and R¹⁴ is independently hydrogen, (CH₂)ₙ—CHO, (CH₂)ₙ—CO₂R¹⁵ or a C₁-C₆ saturated or unsaturated alkyl group optionally substituted with one or more of —OH and —NH₂,
n is 0, 1, 2 or 3, and
each R¹⁵ is independently hydrogen, lithium, sodium, potassium, magnesium, calcium, a C₁-C₆ saturated or unsaturated alkyl group optionally substituted with one or more of —OH and —NH₂, or a naturally occurring amino acid.

Preferably the photodiagnostic agent is for the fluorescent or phosphorescent detection of the said diseases. Preferably the photodiagnostic agent is for the fluorescent or phosphorescent detection and quantification of the said diseases.

The photodiagnostic agent of the first aspect of the present invention may be used for the detection of acne, Aids, viral hepatitis, diabetic retinopathy, infection with sars virus, coronary artery stenosis, carotid artery stenosis, intermittent claudication, Asian (chicken) flu virus, cervical dysplasia, or cancer of the blood, cervix, naso-pharynx, trachea, larynx, bronchi, bronchioles, bladder, esophagus, stomach, rectum, colon, prostate, hollow organs, bile duct, ureter, kidney, uterus, vaginal or other female adnexa, or atherosclerosis, multiple sclerosis, diabetes, arthritis, rheumatoid arthritis, a fungal, viral, chlamydial, bacterial, nanobacterial or parasitic infectious disease, HIV, hepatitis, herpes simplex, herpes zoster, psoriasis, a cardiovascular disease, or a dermatological condition. Preferably the photodiagnostic agent may be used for the detection of acne, Aids, viral hepatitis, diabetic retinopathy, infection with sars virus, coronary artery stenosis, carotid artery stenosis, intermittent claudication, Asian (chicken) flu virus, cervical dysplasia, or cancer of the blood, cervix, naso-pharynx, trachea, larynx, bronchi, bronchioles, bladder, esophagus, stomach, rectum, colon, prostate, hollow organs, bile duct, ureter, kidney, uterus, vaginal or other female adnexa. Preferably the photodiagnostic agent may be used for the detection of acne, Aids, viral hepatitis, diabetic retinopathy, infection with sars virus, coronary artery stenosis, carotid artery stenosis, intermittent claudication, or Asian (chicken) flu virus.

The medicament or phototherapeutic agent of the first aspect of the present invention may be used for the treatment of acne, Aids, viral hepatitis, diabetic retinopathy, infection with sars virus, coronary artery stenosis, carotid artery stenosis, intermittent claudication, Asian (chicken) flu virus, cervical dysplasia or cancer of the blood, cervix, naso-pharynx, trachea, larynx, bronchi, bronchioles, bladder, esophagus, stomach, rectum, colon, prostate, hollow organs, bile duct, ureter, kidney, uterus, vaginal or other female adnexa. Preferably the medicament or phototherapeutic agent may be used for the treatment of acne, Aids, viral hepatitis, diabetic retinopathy, infection with sars virus, coronary artery stenosis, carotid artery stenosis, intermittent claudication, or Asian (chicken) flu virus.

Preferably the medicament, phototherapeutic agent or photodiagnostic agent of the first aspect of the present invention is for the treatment or detection of early cancer. For the purposes of the present application, the term “early cancer” means carcinoma in situ or small areas of cancer that are invisible to the naked eye and that are present in such small amounts, typically less than 5 mm, 3 mm or even 1 mm in diameter, that are difficult to detect with traditional detection methods.

Preferably the medicament, phototherapeutic agent or photodiagnostic agent of the first aspect of the present invention is adapted for administration simultaneous with or prior to administration of irradiation or sound. More preferably the medicament, phototherapeutic agent or photodiagnostic agent is adapted for administration prior to administration of irradiation.

Typically the medicament or phototherapeutic agent is administered 10 to 100 hours before the irradiation, more typically 50 to 90 hours before the irradiation, more typically about 72 hours before the irradiation. This delay between administration of the medicament or phototherapeutic agent and the irradiation allows for the medicament or phototherapeutic agent to clear from normal tissue and skin. Typically the photodiagnostic agent is administered 3 to 60 hours before the irradiation, more typically 8 to 40 hours before the irradiation. Less delay is required with photodiagnosis than with phototherapy, because less agent is required for the photodiagnosis process. Because the medicament or phototherapeutic agent adheres tightly to abnormal tissue for about three to five weeks, the light therapy can be given over a large area without fear of phototoxicity to skin and normal tissue.

The precise wavelength of the irradiation or sound used depends on the compound administered to the human or animal. However, generally the irradiation is electromagnetic radiation with a wavelength in the range of from 500 nm to 1000 nm, preferably from 600 nm to 900 nm, more preferably from 620 nm to 820 nm, and even more preferably from 630 nm to 710 nm. For therapeutic and diagnostic purposes, typically the irradiation or sound is administered for 1 minute to 5 hours, more typically for 2 minutes to 1 hour, even more typically for 5 minutes to 30 minutes.

A second aspect of the present invention is a method of treating acne, Aids, viral hepatitis, diabetic retinopathy, infection with sars virus, coronary artery stenosis, carotid artery stenosis, intermittent claudication, Asian (chicken) flu virus, cervical dysplasia or cancer of the blood, cervix, naso-pharynx, trachea, larynx, bronchi, bronchioles, bladder, esophagus, stomach, rectum, colon, prostate, hollow organs, bile duct, ureter, kidney, uterus, vaginal or other female adnexa, comprising administering a therapeutically effective amount of a compound to a human or animal in need thereof, wherein the compound is of formula 3

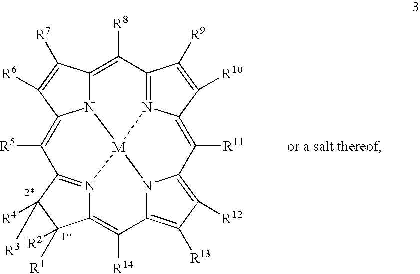

wherein
M is a metal atom in the M(II) oxidation state, a metal halide, a metal oxide or a silicon with two substituents,
each R¹, R², R³, R⁴, R⁵, R⁶, R⁷, R⁸, R⁹, R¹⁰, R¹¹, R¹², R¹³ and R¹⁴ is independently hydrogen, (CH₂)ₙ—CHO, (CH₂)—CO₂R¹⁵ or a C₁-C₆ saturated or unsaturated alkyl group optionally substituted with one or more of —OH and —NH₂,
n is 0, 1, 2 or 3, and
each R¹⁵ is independently hydrogen, lithium, sodium, potassium, magnesium, calcium, a C₁-C₆ saturated or unsaturated alkyl group optionally substituted with one or more of —OH and —NH₂, or a naturally occurring amino acid.

Preferably the human or animal is further subjected to irradiation or sound.

The second aspect of the present invention further provides a method of photodynamic therapy or cytoluminescent therapy of a human or animal disease, comprising administering a therapeutically effective amount of a compound to a human or animal in need thereof and subjecting the human or animal to irradiation or sound, wherein the human or animal disease is acne, Aids, viral hepatitis, diabetic retinopathy, infection with sars virus, coronary artery stenosis, carotid artery stenosis, intermittent claudication, Asian (chicken) flu virus, cervical dysplasia or cancer of the blood, cervix, naso-pharynx, trachea, larynx, bronchi, bronchioles, bladder, esophagus, stomach, rectum, colon, prostate, hollow organs, bile duct, ureter, kidney, uterus, vaginal or other female adnexa, and wherein the compound is of formula 3

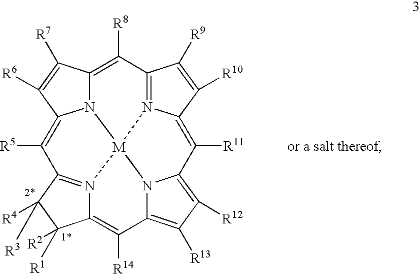

wherein
M is a metal atom in the M(II) oxidation state, a metal halide, a metal oxide or a silicon with two substituents,
each R¹, R², R³, R⁴, R⁵, R⁶, R⁷, R⁸, R⁹, R¹⁰, R¹¹, R¹², R¹³ and R¹⁴ is independently hydrogen, (CH₂)ₙ—CHO, (CH₂)ₙ—CO₂R¹⁵ or a C₁-C₆ saturated or unsaturated alkyl group optionally substituted with one or more of —OH and —NH₂,
n is 0, 1, 2 or 3, and
each R¹⁵ is independently hydrogen, lithium, sodium, potassium, magnesium, calcium, a C₁-C₆ saturated or unsaturated alkyl group optionally substituted with one or more of —OH and —NH₂, or a naturally occurring amino acid.

The second aspect of the present invention further provides a method of photodiagnosis for the detection of acne, Aids, viral hepatitis, diabetic retinopathy, infection with sars virus, coronary artery stenosis, carotid artery stenosis, intermittent claudication, Asian (chicken) flu virus, cervical dysplasia, or cancer of the blood, cervix, naso-pharynx, trachea, larynx, bronchi, bronchioles, bladder, esophagus, stomach, rectum, colon, prostate, hollow organs, bile duct, ureter, kidney, uterus, vaginal or other female adnexa, or atherosclerosis, multiple sclerosis, diabetes, arthritis, rheumatoid arthritis, a fungal, viral, chlamydial, bacterial, nanobacterial or parasitic infectious disease, HIV, hepatitis, herpes simplex, herpes zoster, psoriasis, a cardiovascular disease, or a dermatological condition, in a human or animal, comprising administering a compound to a human or animal and subjecting the human or animal to irradiation or sound, wherein the compound is of formula 3

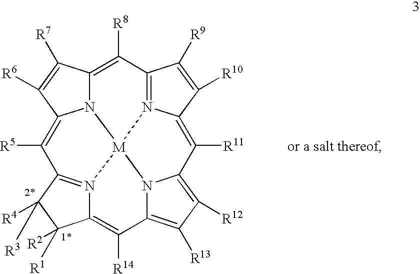

wherein
M is a metal atom in the M(II) oxidation state, a metal halide, a metal oxide or a silicon with two substituents,
each R¹, R², R³, R⁴, R⁵, R⁶, R⁷, R⁸, R⁹, R¹⁰, R¹¹, R¹², R¹³ and R¹⁴ is independently hydrogen, (CH₂)ₙ—CHO, (CH₂)ₙ—CO₂R¹⁵ or a C₁-C₆ saturated or unsaturated alkyl group optionally substituted with one or more of —OH and —NH₂,
n is 0, 1, 2 or 3, and
each R¹⁵ is independently hydrogen, lithium, sodium, potassium, magnesium, calcium, a C₁-C₆ saturated or unsaturated alkyl group optionally substituted with one or more of —OH and —NH₂, or a naturally occurring amino acid.

Preferably the photodiagnosis is for the fluorescent or phosphorescent detection of the said diseases. Preferably the photodiagnosis is for the fluorescent or phosphorescent detection and quantification of the said diseases.

The method of photodiagnosis of the second aspect of the present invention may be used for the detection of acne, Aids, viral hepatitis, diabetic retinopathy, infection with sars virus, coronary artery stenosis, carotid artery stenosis, intermittent claudication, Asian (chicken) flu virus, cervical dysplasia, or cancer of the blood, cervix, naso-pharynx, trachea, larynx, bronchi, bronchioles, bladder, esophagus, stomach, rectum, colon, prostate, hollow organs, bile duct, ureter, kidney, uterus, vaginal or other female adnexa, or atherosclerosis, multiple sclerosis, diabetes, arthritis, rheumatoid arthritis, a fungal, viral, chlamydial, bacterial, nanobacterial or parasitic infectious disease, HIV, hepatitis, herpes simplex, herpes zoster, psoriasis, a cardiovascular disease, or a dermatological condition. Preferably the method of photodiagnosis may be used for the detection of acne, Aids, viral hepatitis, diabetic retinopathy, infection with sars virus, coronary artery stenosis, carotid artery stenosis, intermittent claudication, Asian (chicken) flu virus, cervical dysplasia, or cancer of the blood, cervix, naso-pharynx, trachea, larynx, bronchi, bronchioles, bladder, esophagus, stomach, rectum, colon, prostate, hollow organs, bile duct, ureter, kidney, uterus, vaginal or other female adnexa. Preferably the method of photodiagnosis may be used for the detection of acne, Aids, viral hepatitis, diabetic retinopathy, infection with sars virus, coronary artery stenosis, carotid artery stenosis, intermittent claudication, or Asian (chicken) flu virus.

The method of treatment or therapy of the second aspect of the present invention may be used for the treatment of acne, Aids, viral hepatitis, diabetic retinopathy, infection with sars virus, coronary artery stenosis, carotid artery stenosis, intermittent claudication, Asian (chicken) flu virus, cervical dysplasia or cancer of the blood, cervix, naso-pharynx, trachea, larynx, bronchi, bronchioles, bladder, esophagus, stomach, rectum, colon, prostate, hollow organs, bile duct, ureter, kidney, uterus, vaginal or other female adnexa. Preferably the method of treatment or therapy may be used for the treatment of acne, Aids, viral hepatitis, diabetic retinopathy, infection with sars virus, coronary artery stenosis, carotid artery stenosis, intermittent claudication, or Asian (chicken) flu virus.

Preferably the methods of the second aspect of the present invention are for the treatment or detection of early cancer. For the purposes of the present application, the term “early cancer” means carcinoma in situ or small areas of cancer that are invisible to the naked eye and that are present in such small amounts, typically less than 5 mm, 3 mm or even 1 mm in diameter, that are difficult to detect with traditional detection methods.

Preferably, in any of the methods of the second aspect of the present invention, the human or animal is subjected to irradiation or sound simultaneously with or after administration of the compound of formula 3 or a salt thereof. More preferably the human or animal is subjected to irradiation after administration of the compound of formula 3 or a salt thereof.

For therapeutic methods, typically the compound is administered 10 to 100 hours before the irradiation, more typically 50 to 90 hours before the irradiation, more typically about 72 hours before the irradiation. This delay between administration of the compound and the irradiation allows for the compound to clear from normal tissue and skin. For diagnostic methods, typically the compound is administered 3 to 60 hours before the irradiation, more typically 8 to 40 hours before the irradiation. Less delay is required with photodiagnosis than with phototherapy, because less compound is required for the photodiagnosis process. Because the compound adheres tightly to abnormal tissue for about three to five weeks, the light therapy can be given over a large area without fear of phototoxicity to skin and normal tissue.

The precise wavelength of the irradiation or sound used depends on the compound administered to the human or animal. However, generally the irradiation is electromagnetic radiation with a wavelength in the range of from 500 nm to 1000 nm, preferably from 600 nm to 900 nm, more preferably from 620 nm to 820 nm, and even more preferably from 630 nm to 710 nm. For therapeutic and diagnostic purposes, typically the irradiation or sound is administered for 1 minute to 5 hours, more typically for 2 minutes to 1 hour, even more typically for 5 minutes to 30 minutes.

The compound used in the first or second aspect of the present invention may be immobilized on a protein, a polypeptide, a polymer or activated charcoal. Preferably the compound is immobilized in monomer form. Preferably the protein is serum humane albumin (SHA) or bovine serum albumin (BSA), more preferably serum humane albumin (SHA). Preferably the polypeptide is a low molecular weight polypeptide, more preferably polylysine or polyasparagine. Preferably the polymer is polyvinylpyrrolidone (PVP).

The compound used in the first or second aspect of the present invention may be linked to a photosensitive material such as a nano-dot to enhance the luminescence and diagnostic capabilities and sensitivity for deep seated turnouts or pathology. Nano-dots, also called quantum-dots, are nanometer scale particles that are neither small molecules nor bulk solids. Their composition and small size (a few hundred to a few thousand atoms) give these dots extraordinary optical properties that can be readily customized by changing the size and composition of the dots. Quantum dots fluoresce intensely with illumination.

M of the compound used in the first or second aspect of the present invention is a metal atom in the M(II) oxidation state, a metal halide, a metal oxide or a silicon with two substituents. The metal halide may be a metal fluoride, chloride, bromide, iodide or a mixture thereof. The silicon with two substituents may be SiR₂ where R is a C₁-C₈ saturated or unsaturated alkyl group.

Preferably, in particular if the compound is immobilized, M is Mg, Ca, Ti, V, Nb, Cr, Mo, Mn, Tc, Fe, Ru, Co, Rh, Ni, Pd, Pt, Cu, Ag, Au, Zn, Cd, Hg, Al, Ga, In, Ge, Sn, Pb, a lanthanide, or SiR₂ where R is a C₁-C₈ saturated or unsaturated alkyl group. Preferably M is Mg, Ca, Ti, V, Nb, Cr, Mo, Mn, Tc, Fe, Ru, Co, Rh, Ni, Pd, Pt, Cu, Ag, Au, Zn, Cd, Hg, Al, Ga, In, Ge, Sn, Pb or a lanthanide. Preferably M is Zn, Cu, Cd, Ca, Mn, Au or Co. Preferably M is Zn, Cd, Ca, Mn, Au or Co. More preferably M is Zn.

Preferably, in particular if the compound is not immobilized, M is Ca, Ti, V, Nb, Cr, Mo, Mn, Tc, Ru, Co, Rh, Ni, Pd, Pt, Ag, Au, Zn, Cd, Hg, Al, Ga, In, Ge, Pb, a lanthanide, or SiR₂ where R is a C₁-C₈ saturated or unsaturated alkyl group. Preferably M is Zn, Cd, Ca, Mn, Au or Co. More preferably M is Zn.

For the purposes of this invention, a “salt” of a compound used in the first or second aspect of the present invention is formed between a carboxylic acid functionality of the compound and a suitable cation. Suitable cations include, but are not limited to lithium, sodium, potassium, magnesium, calcium and ammonium. Preferably the salt is a pharmaceutically acceptable salt. The salt may be a mono-, di- or tri-salt. Preferably the salt is a mono- or di-lithium, sodium, potassium, magnesium, calcium or ammonium salt. More preferably the salt is a mono- or di-sodium salt.

The compound used in the first or second aspect of the present invention comprises groups R¹ to R¹⁴. Preferably each R¹, R², R³, R⁴, R⁵, R⁶, R⁷, R⁸, R⁹, R¹⁰, R¹¹, R¹², R¹³ and R¹⁴ is independently hydrogen, methyl, ethyl, propyl, allyl, CO₂H, CH₂CO₂H or (CH₂)₂CO₂H. Preferably R¹ and R³ are hydrogen. Preferably R⁵, R⁸ and R¹¹ are hydrogen.

The compound used in the first or second aspect of the present invention may comprise group R¹⁵. Preferably R¹⁵ is hydrogen, sodium, a C₁-C₆ saturated or unsaturated alkyl group or a naturally occurring amino acid, such as aspartic acid or lysine.

The compound used in the first or second aspect of the present invention has at least two chiral centres, 1* and 2*, and can therefore exist in the form of at least four stereoisomers. The present invention embraces the use of all of these stereoisomers and mixtures thereof. Mixtures of the stereoisomers can be resolved by conventional methods, for example, chiral chromatography, fractional recrystallisation, derivatisation to form diastereomers and subsequent resolution, and resolution using enzymes. Alternatively, the compound can be prepared directly in substantially enantiomerically pure form by enantioselective or stereoselective synthesis.

The compound used in the first or second aspect of the present invention preferably comprises at least 95% of one enantiomer, preferably at least 98% of one enantiomer, and more preferably at least 99% of one enantiomer. Preferably the compound is substantially enantiomerically pure, which is defined for the purposes of the present invention as meaning that the compound comprises at least 99% of one enantiomer.

Preferably R¹ and R³ are hydrogen, and R¹ is in the down-configuration and R³ is in the up-configuration in formula 3 as shown. More preferably R¹ and R³ are hydrogen, R² is (CH₂)₂CO₂H, R⁴ is CO₂H, and chiral centres 1* and 2* are in the (S)-configuration.

In the most preferred embodiment, the compound used in the first or second aspect of the present invention is of formula 2

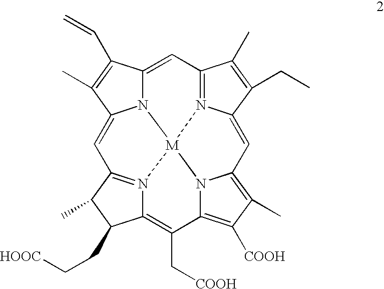

The compound used in the first and second aspects of the present invention may be used together with a pharmaceutically acceptable carrier or diluent.

Preferably the compound is in a form suitable for oral, parental (including intravenous, subcutaneous, intramuscular, intradermal, intratracheal, intraperitoneal, intraarticular, intraabdominal, intracranial and epidural), transdermal, airway (aerosol), rectal, vaginal or topical (including buccal, mucosal and sublingual) administration, most preferably in a form suitable for oral or parental administration.

For oral administration, the compound is preferably provided in the form of a tablet, capsule, hard or soft gelatine capsule, caplet, troche or lozenge, as a powder or granules, or as an aqueous solution, suspension or dispersion.

Alternatively, the compound may be in a form suitable for parental, in particular intravenous, administration, in which case the pharmaceutical composition is preferably an aqueous solution or suspension having a pH of from 6 to 8.5.

For therapeutic purposes, the compound is preferably in a form suitable for providing 0.01 to 10 mg/kg/day of a compound of formula 3 or a salt thereof, more preferably 0.1 to 5 mg/kg/day, and even more preferably about 2 mg/kg/day.

For diagnostic purposes, the compound is preferably in a form suitable for providing 0.1 to 20 mg of a compound of formula 3 or a salt thereof per diagnosis, more preferably 0.5 to 10 mg, and even more preferably 1 to 3 mg.

Preferably the human or animal to be treated or diagnosed in the first or second aspect of the present invention is a human.

A third aspect of the present invention is a method of cold sterilising a surgical or other device, comprising the steps of: providing a compound on the device and subjecting the device to irradiation or sound, wherein the compound is of formula 3

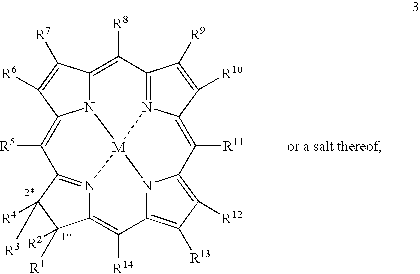

wherein
M is a metal atom in the M(II) oxidation state, a metal halide, a metal oxide or a silicon with two substituents,

each R¹, R², R³, R⁴, R⁵, R⁶, R⁷, R⁸, R⁹, R¹⁰, R¹¹, R¹², R¹³ and R¹⁴ is independently hydrogen, (CH₂)ₙ—CHO, (CH₂)ₙ—CO₂R¹⁵ or a C₁-C₆ saturated or unsaturated alkyl group optionally substituted with one or more of —OH and —NH₂,
n is 0, 1, 2 or 3, and
each R¹⁵ is independently hydrogen, lithium, sodium, potassium, magnesium, calcium, a C₁-C₆ saturated or unsaturated alkyl group optionally substituted with one or more of —OH and —NH₂, or a naturally occurring amino acid.

Preferably the device is subjected to irradiation or sound simultaneously with or after provision of the compound of formula 3 or a salt thereof on the device. More preferably the device is subjected to irradiation after provision of the compound of formula 3 or a salt thereof on the device. Typically the compound is provided on the device 1 to 60 minutes before the irradiation, more typically 1 to 45 minutes before the irradiation, even more typically 2 to 30 minutes before the irradiation.

The precise wavelength of the irradiation or sound used depends on the compound used for the cold sterilisation. However, generally the irradiation is electromagnetic radiation with a wavelength in the range of from 500 nm to 1000 nm, preferably from 600 nm to 900 nm, more preferably from 620 nm to 820 nm, and even more preferably from 630 nm to 710 nm. Typically the device is subjected to irradiation or sound for 1 minute to 24 hours, more typically for 10 minutes to 5 hours.

The compound used in the third aspect of the present invention may be immobilized on a protein, a polypeptide, a polymer or activated charcoal. Preferably the compound is immobilized in monomer form. Preferably the protein is serum humane albumin (SHA) or bovine serum albumin (BSA), more preferably serum humane albumin (SHA). Preferably the polypeptide is a low molecular weight polypeptide, more preferably polylysine or polyasparagine. Preferably the polymer is polyvinylpyrrolidone (PVP).

M of the compound used in the third aspect of the present invention is a metal atom in the M(II) oxidation state, a metal halide, a metal oxide or a silicon with two substituents. The metal halide may be a metal fluoride, chloride, bromide, iodide or a mixture thereof. The silicon with two substituents may be SiR₂ where R is a C₁-C₈ saturated or unsaturated alkyl group.

Preferably, in particular if the compound is immobilized, M is Mg, Ca, Ti, V, Nb, Cr, Mo, Mn, Tc, Fe, Ru, Co, Rh, Ni, Pd, Pt, Cu, Ag, Au, Zn, Cd, Hg, Al, Ga, In, Ge, Sn, Pb, a lanthanide, or SiR₂ where R is a C₁-C₈ saturated or unsaturated alkyl group. Preferably M is Mg, Ca, Ti, V, Nb, Cr, Mo, Mn, Tc, Fe, Ru, Co, Rh, Ni, Pd, Pt, Cu, Ag, Au, Zn, Cd, Hg, Al, Ga, In, Ge, Sn, Pb or a lanthanide. Preferably M is Zn, Cu, Cd, Ca, Mn, Au or Co. Preferably M is Zn, Cd, Ca, Mn, Au or Co. More preferably M is Zn.

Preferably, in particular if the compound is not immobilized, M is Ca, Ti, V, Nb, Cr, Mo, Mn, Tc, Ru, Co, Rh, Ni, Pd, Pt, Ag, Au, Zn, Cd, Hg, Al, Ga, In, Ge, Pb, a lanthanide, or SiR₂ where R is a C₁-C₈ saturated or unsaturated alkyl group. Preferably M is Zn, Cd, Ca, Mn, Au or Co. More preferably M is Zn.

For the purposes of this invention, a “salt” of a compound used in the third aspect of the present invention is formed between a carboxylic acid functionality of the compound and a suitable cation. Suitable cations include, but are not limited to lithium, sodium, potassium, magnesium, calcium and ammonium. Preferably the salt is a pharmaceutically acceptable salt. The salt may be a mono-, di- or tri-salt. Preferably the salt is a mono- or di-lithium, sodium, potassium, magnesium, calcium or ammonium salt. More preferably the salt is a mono- or di-sodium salt.

The compound used in the third aspect of the present invention comprises groups R¹ to R¹⁴. Preferably each R¹, R², R³, R⁴, R⁵, R⁶, R⁷, R⁸, R⁹, R¹⁰, R¹¹, R¹², R¹³ and R¹⁴ is independently hydrogen, methyl, ethyl, propyl, allyl, CO₂H, CH₂CO₂H or (CH₂)₂CO₂H. Preferably R¹ and R³ are hydrogen. Preferably R⁵, R⁸ and R¹¹ are hydrogen.

The compound used in the third aspect of the present invention may comprise group R¹⁵. Preferably R¹⁵ is hydrogen, sodium, a C₁-C₆ saturated or unsaturated alkyl group or a naturally occurring amino acid, such as aspartic acid or lysine.

The compound used in the third aspect of the present invention has at least two chiral centres, 1* and 2*, and can therefore exist in the form of at least four stereoisomers. The present invention embraces the use of all of these stereoisomers and mixtures thereof. Mixtures of the stereoisomers can be resolved by conventional methods, for example, chiral chromatography, fractional recrystallisation, derivatisation to form diastereomers and subsequent resolution, and resolution using enzymes. Alternatively, the compound can be prepared directly in substantially enantiomerically pure form by enantioselective or stereoselective synthesis.

The compound used in the third aspect of the present invention preferably comprises at least 95% of one enantiomer, preferably at least 98% of one enantiomer, and more preferably at least 99% of one enantiomer. Preferably the compound is substantially enantiomerically pure, which is defined for the purposes of the present invention as meaning that the compound comprises at least 99% of one enantiomer.

Preferably R¹ and R³ are hydrogen, and R¹ is in the down-configuration and R³ is in the up-configuration in formula 3 as shown. More preferably R¹ and R³ are hydrogen, R² is (CH₂)₂CO₂H, R⁴ is CO₂H, and chiral centres 1* and 2* are in the (S)-configuration.

In the most preferred embodiment, the compound used in the third aspect of the present invention is of formula 2

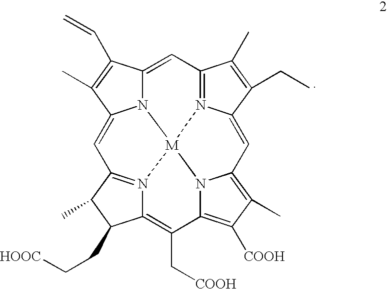

The compound used in the third aspect of the present invention may be used together with a carrier or diluent.

A fourth aspect of the present invention is a compound of formula 3

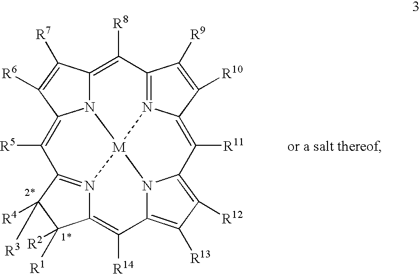

wherein
M is a metal atom in the M(II) oxidation state, a metal halide, a metal oxide or a silicon with two substituents,
each R¹, R², R³, R⁴, R⁵, R⁶, R⁷, R⁸, R⁹, R¹⁰, R¹¹, R¹², R¹³ and R¹⁴ is independently hydrogen, (CH₂)ₙ—CHO, (CH₂)ₙ—CO₂R¹⁵ or a C₁-C₆ saturated or unsaturated alkyl group optionally substituted with one or more of —OH and —NH₂,
n is 0, 1, 2 or 3, and
each R's is independently hydrogen, lithium, sodium, potassium, magnesium, calcium, a C₁-C₆ saturated or unsaturated alkyl group optionally substituted with one or more of —OH and —NH₂, or a naturally occurring amino acid,
wherein the compound is linked or attached to a magnetic element.

Preferably the magnetic element is Gd, Fe or Mn.

The compound of the fourth aspect of the present invention may be used as an MRI enhancer. Magnetic resonance imaging (MRI) is an imaging technique used primarily in medical settings to produce high quality images of the inside of the human body. MRI is based on the principles of nuclear magnetic resonance (NMR). In effect, MRI measures differentials in magnetic strengths of different tissues.

With the compound of the fourth aspect of the present invention, comprising the magnetic element, selectively attaching to for example tumours, the MRI more accurately identifies the malignant tissue. The compound of the fourth aspect of the present invention need not be photoactivated by illumination or sound for the MRI scan. The compound of the fourth aspect of the present invention acts as a carrier to concentrate the magnetic element in tumour or other diseased tissue, thereby making the tissue highly visible on the MRI scan in a way that is not possible with present technology.

The compound of the fourth aspect of the present invention may be immobilized on a protein, a polypeptide, a polymer or activated charcoal. Preferably the compound is immobilized in monomer form. Preferably the protein is serum humane albumin (SHA) or bovine serum albumin (BSA), more preferably serum humane albumin (SHA). Preferably the polypeptide is a low molecular weight polypeptide, more preferably polylysine or polyasparagine. Preferably the polymer is polyvinylpyrrolidone (PVP).

M of the compound of the fourth aspect of the present invention is a metal atom in the M(II) oxidation state, a metal halide, a metal oxide or a silicon with two substituents. The metal halide may be a metal fluoride, chloride, bromide, iodide or a mixture thereof. The silicon with two substituents may be SiR₂ where R is a C₁-C₈ saturated or unsaturated alkyl group.

Preferably, in particular if the compound is immobilized, M is Mg, Ca, Ti, V, Nb, Cr, Mo, Mn, Tc, Fe, Ru, Co, Rh, Ni, Pd, Pt, Cu, Ag, Au, Zn, Cd, Hg, Al, Ga, In, Ge, Sn, Pb, a lanthanide, or SiR₂ where R is a C₁-C₈ saturated or unsaturated alkyl group. Preferably M is Mg, Ca, Ti, V, Nb, Cr, Mo, Mn, Tc, Fe, Ru, Co, Rh, Ni, Pd, Pt, Cu, Ag, Au, Zn, Cd, Hg, Al, Ga, In, Ge, Sn, Pb or a lanthanide. Preferably M is Zn, Cu, Cd, Ca, Mn, Au or Co. Preferably M is Zn, Cd, Ca, Mn, Au or Co. More preferably M is Zn.

Preferably, in particular if the compound is not immobilized, M is Ca, Ti, V, Nb, Cr, Mo, Mn, Tc, Ru, Co, Rh, Ni, Pd, Pt, Ag, Au, Zn, Cd, Hg, Al, Ga, In, Ge, Pb, a lanthanide, or SiR₂ where R is a C₁-C₈ saturated or unsaturated alkyl group. Preferably M is Zn, Cd, Ca, Mn, Au or Co. More preferably M is Zn.

For the purposes of this invention, a “salt” of a compound of the fourth aspect of the present invention is formed between a carboxylic acid functionality of the compound and a suitable cation. Suitable cations include, but are not limited to lithium, sodium, potassium, magnesium, calcium and ammonium. Preferably the salt is a pharmaceutically acceptable salt. The salt may be a mono-, di- or tri-salt. Preferably the salt is a mono- or di-lithium, sodium, potassium, magnesium, calcium or ammonium salt. More preferably the salt is a mono- or di-sodium salt.

The compound of the fourth aspect of the present invention comprises groups R¹ to R¹⁴. Preferably each R¹, R², R³, R⁴, R⁵, R⁶, R⁷, R⁸, R⁹, R¹⁰, R¹¹, R¹², R¹³ and R¹⁴ is independently hydrogen, methyl, ethyl, propyl, allyl, CO₂H, CH₂CO₂H or (CH₂)₂CO₂H. Preferably R¹ and R³ are hydrogen. Preferably R⁵, R⁸ and R¹¹ are hydrogen.

The compound of the fourth aspect of the present invention may comprise group R¹⁵. Preferably R¹⁵ is hydrogen, sodium, a C₁-C₆ saturated or unsaturated alkyl group or a naturally occurring amino acid, such as aspartic acid or lysine.

The compound of the fourth aspect of the present invention has at least two chiral centres, 1* and 2*, and can therefore exist in the form of at least four stereoisomers. The present invention embraces the use of all of these stereoisomers and mixtures thereof. Mixtures of the stereoisomers can be resolved by conventional methods, for example, chiral chromatography, fractional recrystallisation, derivatisation to form diastereomers and subsequent resolution, and resolution using enzymes. Alternatively, the compound can be prepared directly in substantially enantiomerically pure form by enantioselective or stereoselective synthesis.

The compound of the fourth aspect of the present invention preferably comprises at least 95% of one enantiomer, preferably at least 98% of one enantiomer, and more preferably at least 99% of one enantiomer. Preferably the compound is substantially enantiomerically pure, which is defined for the purposes of the present invention as meaning that the compound comprises at least 99% of one enantiomer.

Preferably R¹ and R³ are hydrogen, and R¹ is in the down-configuration and R³ is in the up-configuration in formula 3 as shown. More preferably R¹ and R³ are hydrogen, R² is (CH₂)₂CO₂H, R⁴ is CO₂H, and chiral centres 1* and 2* are in the (S)-configuration.

In the most preferred embodiment, the compound of the fourth aspect of the present invention is of formula 2

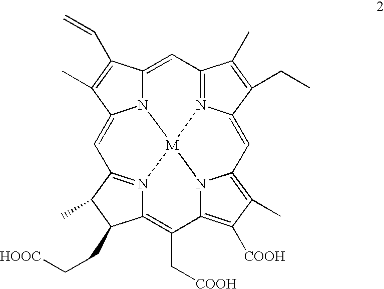

The compound of the fourth aspect of the present invention may be used together with a pharmaceutically acceptable carrier or diluent.

Preferably the compound is in a form suitable for oral, parental (including intravenous, subcutaneous, intramuscular, intradermal, intratracheal, intraperitoneal, intraarticular, intraabdominal, intracranial and epidural), transdermal, airway (aerosol), rectal, vaginal or topical (including buccal, mucosal and sublingual) administration, most preferably in a form suitable for oral or parental administration.

For oral administration, the compound is preferably provided in the form of a tablet, capsule, hard or soft gelatine capsule, caplet, troche or lozenge, as a powder or granules, or as an aqueous solution, suspension or dispersion.

Alternatively, the compound may be in a form suitable for parental, in particular intravenous, administration, in which case the pharmaceutical composition is preferably an aqueous solution or suspension having a pH of from 6 to 8.5.

The compound is preferably in a form suitable for providing 0.1 to 20 mg of a compound of formula 3 or a salt thereof per diagnosis, more preferably 0.5 to 10 mg, and even more preferably 1 to 3 mg.

The fourth aspect of the present invention further provides a method of carrying out an MRI scan, the method comprising using a compound of the fourth aspect of the present invention as an MRI enhancer.

The fourth aspect of the present invention further provides the use of a compound of the fourth aspect of the present invention as an MRI enhancer.

Preferably the MRI scan is carried out on a human or animal, more preferably a human.

### BRIEF DESCRIPTION OF THE DRAWINGS

FIG. 1 shows the absorption spectra of (1) chlorine-e6 (λₘₐₓ=656 nm), (2) Zn-chlorine-e6 complex (λₘₐₓ=632 nm), and (3) Zn-chlorine-e6 complex immobilized on SHA (λₘₐₓ=636 nm), all in water.

FIG. 2 shows the absorption spectrum of chlorine-e6 (λₘₐₓ=402, 502 and 656 nm) in water.

FIG. 3 shows the absorption spectra of (1) chlorine-e6 (λₘₐₓ=656 nm), (2) chlorine-e6 immobilized on SHA (λₘₐₓ=662 nm), and (3) Zn-chlorine-e6 complex immobilized on SHA (λₘₐₓ=636 nm), all in water.

FIG. 4 shows the absorption spectra of (1) chlorine-e6 (λₘₐₓ=656 nm), (2) Zn-chlorine-e6 complex (λₘₐₓ=632 nm), and (3) Zn-chlorine-e6 complex immobilized on PVP (λₘₐₓ=638 nm), all in water.

FIG. 5 shows the absorption spectra of (1) chlorine-e6 (λₘₐₓ=656 nm), (2) chlorine-e6 immobilized on PVP (λₘₐₓ=662 nm), and (3) Zn-chlorine-e6 complex immobilized on PVP (λₘₐₓ=638 nm), all in water.

FIGS. 6 to 8 show the absorption spectra of Zn-chlorine-e6 complex (λₘₐₓ=414 and 634 nm), Zn-chlorine-e6 complex immobilized on SHA (λₘₐₓ=418 and 636 nm), and Zn-chlorine-e6 complex immobilized on PVP (λₘₐₓ=416 and 638 nm), all in water, respectively.

FIGS. 9 and 10 show the fluorescence spectrum (λₘₐₓ=643 nm) and the fluorescence stimulation spectrum (λₘₐₓ=412 and 607 nm) of Zn-chlorine-e6 complex in water respectively.

FIGS. 11 and 12 show the fluorescence spectrum (λₘₐₓ=645 nm) and the fluorescence stimulation spectrum (λₘₐₓ=446 and 673 nm) of Zn-chlorine-e6 complex immobilized on SHA in water respectively.

FIGS. 13 and 14 show the fluorescence spectrum (λₘₐₓ=645 nm) and the fluorescence stimulation spectrum (λₘₐₓ=429 and 727 nm) of Zn-chlorine-e6 complex immobilized on PVP in water respectively.

FIGS. 15 and 16 show the fluorescence spectrum (λₘₐₓ=645 nm) and the fluorescence stimulation spectrum (λₘₐₓ=418 and 641 nm) of a biological sample taken from the liquid above the sediment of an ascite tumour taken from an experimental animal (mouse), which had previously been injected intraabdominally with a preparation comprising Zn-chlorine-e6 complex immobilized on SHA.

FIG. 17 shows the absorption spectra of (1) chlorine-e6 (λₘₐₓ=656 nm), (2) chlorine-e6 immobilized on PVP (λₘₐₓ=662 nm), and (3) Cd-chlorine-e6 complex immobilized on PVP (λₘₐₓ=646 nm), all in water.

FIG. 18 shows the absorption spectrum of Cd-chlorine-e6 complex immobilized on PVP (λₘₐₓ=424 and 646 nm) in water.

FIG. 19 shows the absorption spectra of (1) chlorine-e6 (λₘₐₓ=656 nm), (2) chlorine-e6 immobilized on PVP (λₘₐₓ=662 nm), and (3) Cu-chlorine-e6 complex immobilized on PVP (λₘₐₓ=636 nm), all in water.

FIG. 20 shows the absorption spectrum of Cu-chlorine-e6 complex immobilized on PVP (λₘₐₓ=410, 505 and 636 nm) in water.

FIG. 21 shows the results of pharmacokinetic distribution studies. The pharmacokinetic distribution of Zn-chlorine-e6 complex immobilized on SHA over 30 hours in organs, tissues, biological liquids and turnouts (embryocarcinoma) was studied.

### DETAILED DESCRIPTION OF THE INVENTION

There are two routes to compounds of formula 3 and salts thereof.

The first route (see Examples 1 and 2 below) comprises the step of mixing a compound of formula 4, also called chlorine-e6, which is commercially available, with a metal compound in an aqueous solution having a pH ≧9 to yield the compound of formula 3. The compound of formula 3 may be immobilized in monomer form on an immobilizer, such as a protein, a polypeptide, a polymer or activated charcoal, by adding the immobilizer to the compound of formula 3 upon formation.

More specifically, chlorine-e6 is dissolved in an aqueous solution with a pH ≧9. A pH ≧9 can be achieved, for example, by adding ammonia to an aqueous solution.

Then an about equimolar quantity of a metal compound, for example zinc acetate, is added to the reaction mixture. When mixing the solution at about room temperature, chlorine-e6 and the metal ion form a complex. The progress and completion of the complex-formation reaction can be monitored with a spectrophotometer.

On completion of the complex-formation reaction, an about equimolar quantity of an immobilizer such as a protein, a polypeptide, a polymer or activated charcoal, for example serum humane albumin (SHA) or polyvinylpyrrolidone (PVP), is added to the reaction mixture. The solution is mixed at about room temperature until the compound of formula 3 is immobilized on the immobilizer. The progress and completion of the immobilization reaction can be monitored with the help of a spectrophotometer.

The second route (see Examples 3 to 6 below) comprises the steps of (i) mixing a compound of formula 4 with an immobilizer in an aqueous solution having a pH ≧9 to yield an immobilized compound 4, and (ii) adding a metal compound to the immobilized compound 4 to yield an immobilized compound of formula 3. Preferably the compound of formula 4 is immobilized in monomer form on a protein, a polypeptide, a polymer or activated charcoal. The progress and completion of the immobilization and the complex-formation reaction can be monitored with a spectrophotometer.

Thus a water-soluble immobilizer, for example serum humane albumin (SHA) or polyvinylpyrrolidone (PVP), is added to the reaction mixture in an about equimolar quantity relative to chlorine-e6, either before (route 2) or after (route 1) carrying out the complex-formation reaction.

The fact that compounds of formula 3 can be immobilized in monomolecular form on the immobilizer is surprising, since monomeric compounds of formula 3 are not particularly stable in aqueous solution. The quantity of the immobilizer required is defined by the number of sites on the molecule to be immobilized, which is one for compounds of formula 3.

Without wishing to be bound by theory, it is believed that it is the monomer form of the compounds of formula 3, which is the photoactive form, which may be useful as a phototherapeutic or photodiagnostic agent. However, compounds of formula 3, which have not been immobilized, have a tendency to form aggregates (dimers, trimers and oligomers of unknown structure) with unpredictable physical, chemical, photophysical and biological properties, in particular when the compounds of formula 3 are subjected to pHs lower than 9. For example, aggregates of Zn-chlorine-e6 are chemically very stable and attempts to disaggregate the Zn-chlorine-e6 aggregates, for example, by increasing pH, heating, using polar solvents, etc. have failed. Thus the aggregation process is difficult, if not impossible, to reverse. The present invention solves this problem by immobilizing the compounds of formula 3 in monomeric form prior to any aggregation occurring.

The compounds of formula 3 are photosensitizers and therefore useful in pharmaceutical compositions and medicaments for the use in photodynamic therapy. Moreover the photosensitizers of formula 3 can be used as photodiagnostic agents.

The compound, pharmaceutical composition, medicament, phototherapeutic agent or photodiagnostic agent employed in the present invention can be administered by oral, parental (including intravenous, subcutaneous, intramuscular, intradermal, intratracheal, intraperitoneal, intraarticular, intraabdominal, intracranial and epidural), transdermal, airway (aerosol), rectal, vaginal or topical (including buccal, mucosal and sublingual) administration.

For oral administration, the compound, pharmaceutical composition, medicament, phototherapeutic agent or photodiagnostic agent will generally be provided in the form of tablets, capsules, hard or soft gelatine capsules, caplets, troches or lozenges, as a powder or granules, or as an aqueous solution, suspension or dispersion.

Tablets for oral use may include the active ingredient mixed with pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose. Corn starch and alginic acid are suitable disintegrating agents. Binding agents may include starch and gelatine. The lubricating agent, if present, may be magnesium stearate, stearic acid or talc. If desired, the tablets may be coated with a material, such as glyceryl monostearate or glyceryl distearate, to delay absorption in the gastrointestinal tract.

Capsules for oral use include hard gelatine capsules in which the active ingredient is mixed with a solid diluent, and soft gelatine capsules wherein the active ingredient is mixed with water or an oil such as peanut oil, liquid paraffin or olive oil.

Formulations for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active ingredient such carriers as are known in the art to be appropriate.

For parenteral use, the active ingredient will generally be provided in a sterile aqueous solution or suspension, buffered to an appropriate pH and isotonicity. Suitable aqueous vehicles include Ringer's solution and isotonic sodium chloride or glucose. Aqueous suspensions may include suspending agents such as cellulose derivatives, sodium alginate, polyvinylpyrrolidone and gum tragacanth, and a wetting agent such as lecithin. Suitable preservatives for aqueous suspensions include ethyl and n-propyl p-hydroxybenzoate. The active ingredient may also be presented as liposome formulations.

For topical and transdermal administration, the active ingredient will generally be provided in the form of ointments, cataplasms (poultices), pastes, powders, dressings, creams, plasters or patches.

Suitable suspensions and solutions can be used in inhalers for airway (aerosol) administration.

In general, a suitable therapeutic dose will be in the range of 0.01 to 10 mg of the active ingredient per kilogram body weight of the recipient per day, preferably in the range of 0.1 to 5 mg per kilogram body weight per day, more preferably about 2 mg per kilogram body weight per day. The desired dose is preferably presented once a day, but may be dosed as two, three, four or more sub-doses administered at appropriate intervals throughout the day. These sub-doses may be administered in unit dosage forms, for example, containing 1 to 1000 mg, preferably 10 to 800 mg, and most preferably 20 to 500 mg of active ingredient per unit dosage form.

In general, a suitable diagnostic dose will be in the range of 0.1 to 20 mg of the active ingredient per diagnosis, more preferably 0.5 to 10 mg, and even more preferably 1 to 3 mg.

The invention will now be described with reference to the following examples. It will be appreciated that what follows is by way of example only and that modifications to detail may be made whilst still falling within the scope of the invention.

### SYNTHETIC EXPERIMENTAL DETAILS

### Example 1

Ammonia was added to water until the pH of the solution was not less than 9. Then chlorine-e6 (1.0 g) was dissolved in the aqueous solution. An equimolar quantity of zinc acetate (0.22 g) was added and the reaction mixture was stirred for 15 minutes at about 20° C. to achieve the complex-formation reaction. The progress and completion of the reaction was monitored with the help of a spectrophotometer. On completion of the complex-formation reaction, serum humane albumin (SHA) (71 g) was added to the reaction mixture as an immobilizer. On completion of the immobilization reaction, which was monitored with a spectrophotometer, the product of the reaction, Zn-chlorine-e6 complex immobilized on SHA, was purified by dialysis.

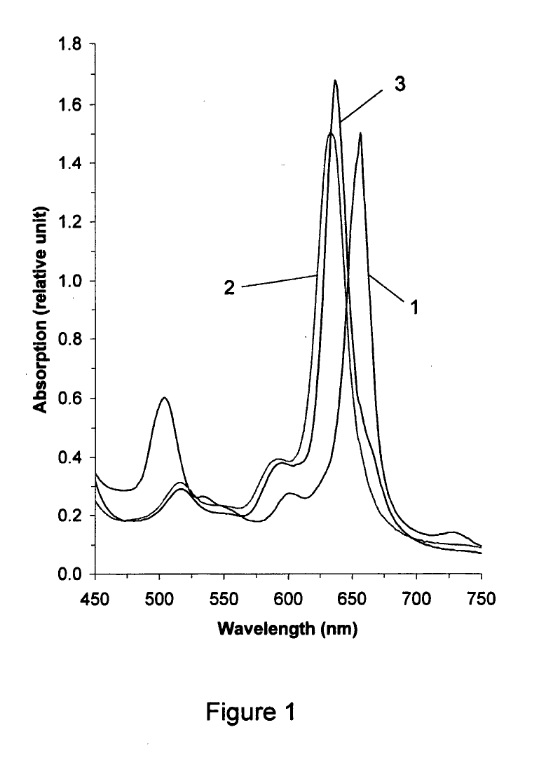

FIG. 1 shows the long-wave region of the visible absorption spectra of (1) the starting material chlorine-e6 (λₘₐₓ=656 nm), (2) Zn-chlorine-e6 complex (λₘₐₓ=632 nm), and (3) Zn-chlorine-e6 complex immobilized on SHA (λₘₐₓ=636 nm), all in water.

As can be seen in FIG. 1, the formation of the Zn-chlorine-e6 complex is accompanied by a 24 nm short-wave shift of the long-wave absorption peak, and the immobilization of Zn-chlorine-e6 on protein causes a 4 nm long-wave shift. Such shifts of the long-wave peak are typical for both complex-formation with metal and immobilization on protein and prove the completeness and purity of the reactions. Moreover, the characteristic absorption peak of chlorine-e6 of medium intensity at λₘₐₓ=502 nm practically disappears for Zn-chlorine-e6, and instead a weak peak at λₘₐₓ=514 nm appears, which also demonstrates the completeness and purity of the reaction.

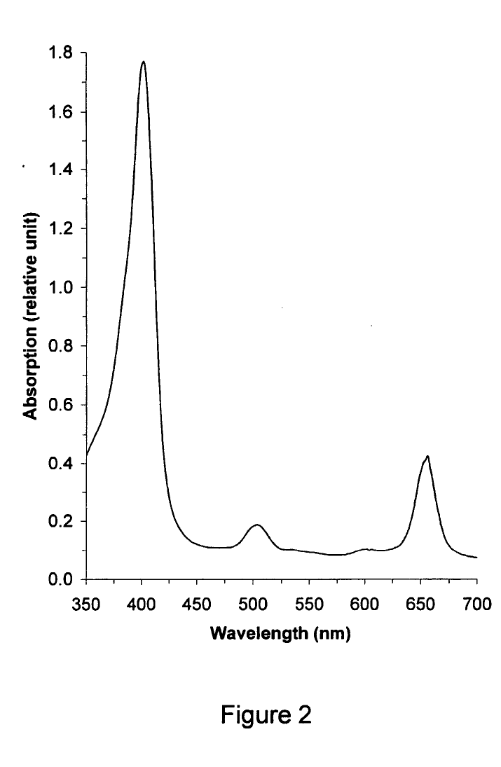

For comparison, FIG. 2 shows the visible absorption spectrum of the starting material chlorine-e6 in water down to 350 nm. The maxima of the main absorption peaks are at λₘₐₓ=402, 502 and 656 nm.

### Example 2

The synthesis of immobilized Zn-chlorine-e6 was carried out as described in Example 1, except that as immobilizer polyvinylpyrrolidone (PVP) (62 g) was used instead of SHA.

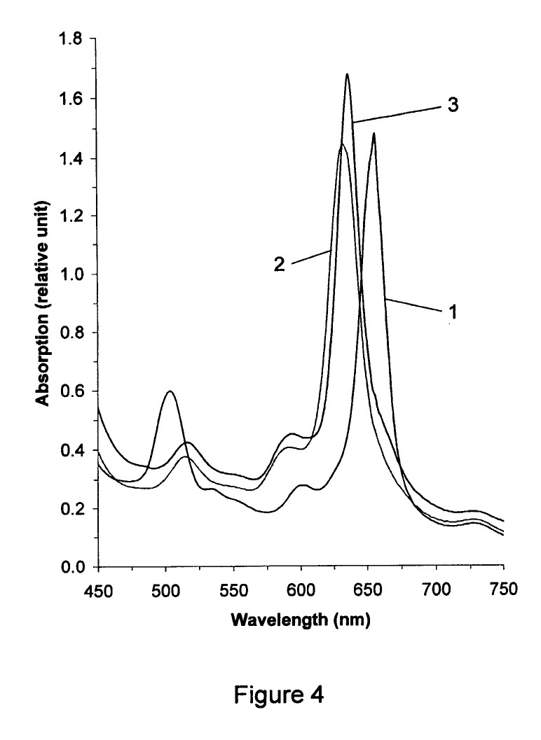

As can be seen in FIG. 4, the spectral picture of the visible absorption spectra of (1) the starting material chlorine-e6 (λₘₐₓ=656 nm), (2) Zn-chlorine-e6 complex (λₘₐₓ=632 nm), and (3) Zn-chlorine-e6 complex immobilized on PVP (λₘₐₓ=638 nm) are practically identical to the ones depicted in FIG. 1. One observes a significant 24 nm short-wave shift of the long-wave peak upon metal complex formation and a small 6 nm long-wave shift upon immobilization on polymer PVP. The medium intensity peak of chlorine-e6 at λₘₐₓ=502 nm practically disappears, when forming the Zn-chlorine-e6 complex. All of these changes prove the completeness of the reactions and the purity and homogeneity of the products obtained.

### Example 3

Ammonia was added to water until the pH of the solution was not less than 9. Then chlorine-e6 (1.0 g) was dissolved in the aqueous solution. An equimolar quantity of SHA (71 g) was added and the reaction mixture was stirred for 17 minutes at about 20° C. to immobilize chlorine-e6 on SHA. Then an equimolar quantity of zinc acetate (0.22 g) was added and the reaction mixture was stirred at room temperature to complex Zn into the chlorine-e6, which was monitored with a spectrophotometer. The product of the reaction, Zn-chlorine-e6 complex immobilized on SHA, was purified by dialysis.

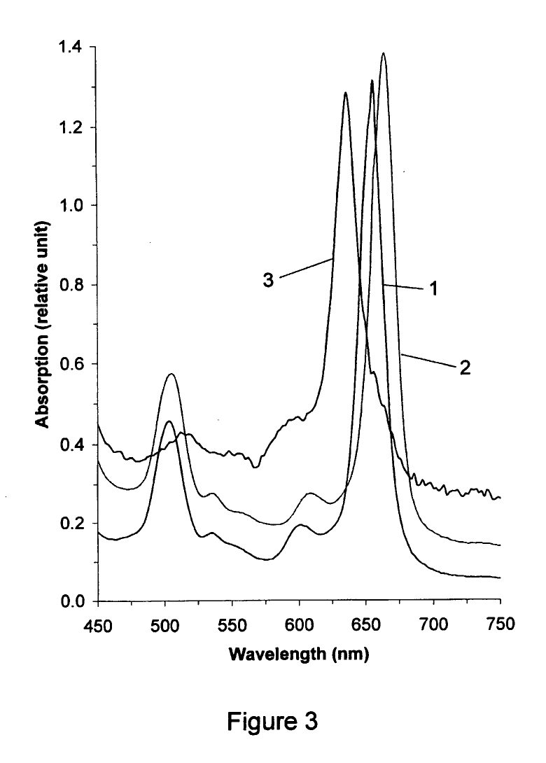

FIG. 3 shows the long-wave region of the visible absorption spectra of (1) the starting material chlorine-e6 (λₘₐₓ=656 nm), (2) chlorine-e6 immobilized on SHA (λₘₐₓ=662 nm), and (3) Zn-chlorine-e6 complex immobilized on SHA (λₘₐₓ=636 nm). Unlike the first method of synthesis (see Example 1), when forming chlorine-e6 immobilized on protein, first a 6 nm long-wave shift of the absorption peak occurs, and then a 26 nm short-wave shift, when forming Zn-chlorine-e6 immobilized on SHA. Such shifts of the absorption peak agree with the properties of the synthesized products and prove the completeness of the reactions and the purity of the products obtained. Moreover, the medium intensity peak of chlorine-e6 (λₘₐₓ=502 nm) is observed in the spectra of chlorine-e6 as well as of chlorine-e6 immobilized on protein, but then it disappears in the spectrum of Zn-chlorine-e6 complex immobilized on protein and gets transformed into a peak at λₘₐₓ=514 nm.

### Example 4

The synthesis of immobilized Zn-chlorine-e6 was carried out as described in Example 3, except that as immobilizer polyvinylpyrrolidone (PVP) (62 g) was used instead of SHA.

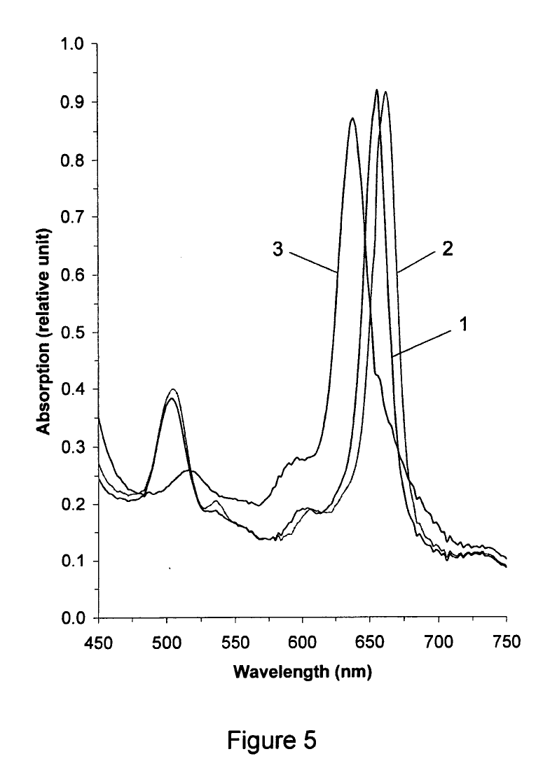

FIG. 5 shows the long-wave region of the visible absorption spectra of (1) the starting material chlorine-e6 (λₘₐₓ=656 nm), (2) chlorine-e6 immobilized on PVP (λₘₐₓ=662 nm), and (3) Zn-chlorine-e6 complex immobilized on PVP (λₘₐₓ=638 nm). As in Example 3, when immobilising chlorine-e6 on PVP, a 6 nm long-wave shift of the absorption peak takes place, and then after introduction of Zn ions into chlorine-e6 and formation of the Zn-chlorine-e6 complex immobilized on PVP, a 24 nm short-wave shift of the absorption peak occurs. These results demonstrate the completeness of the reactions and the purity of the products obtained. They are also evidenced by the behaviour of the medium intensity peak of chlorine-e6 at λₘₐₓ=502 nm, which is present in the spectra of chlorine-e6 as well as of chlorine-e6 immobilized on PVP, but disappears in the spectrum of Zn-chlorine-e6 complex immobilized on PVP.

The fact that the spectra of the products, synthesised by the two different routes discussed above (route 1: Examples 1 and 2, route 2: Examples 3 and 4), are identical proves that the conclusions drawn in the final paragraphs of Examples 1 to 4 are correct.

### Discussion of Further Spectra

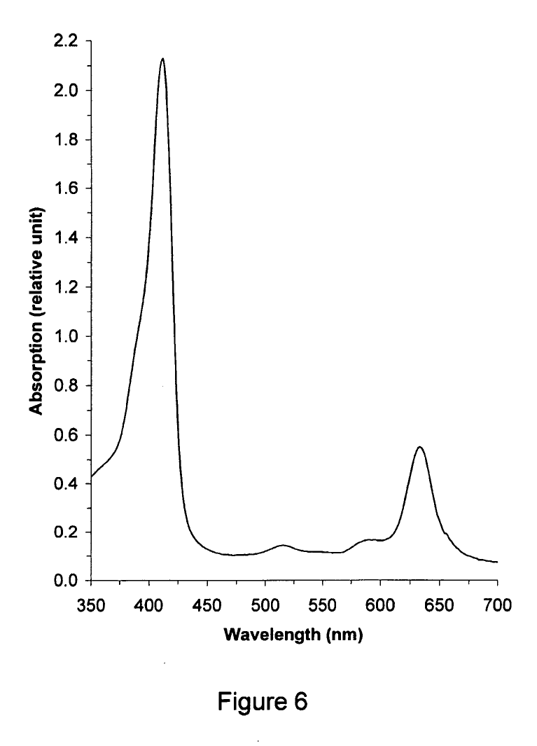

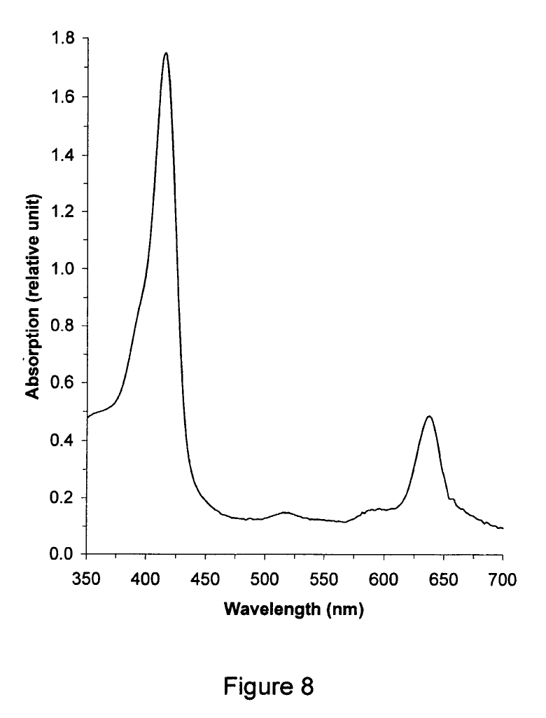

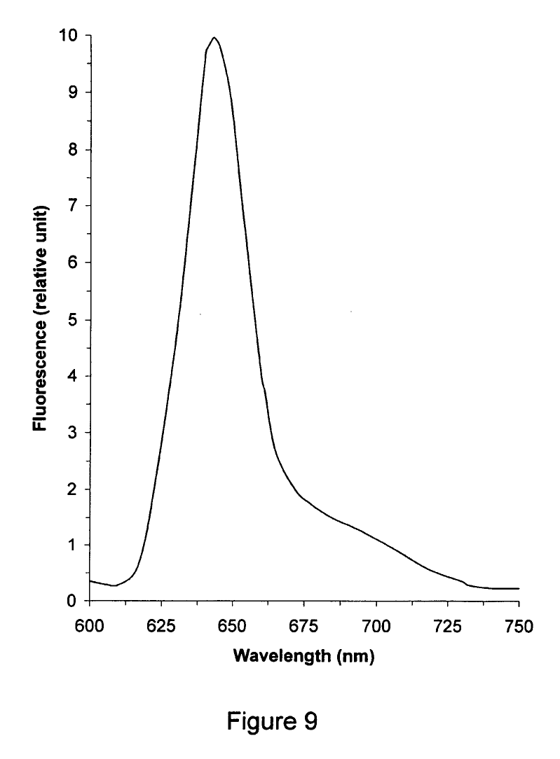

FIGS. 6 to 8, with a spectral range of 350-700 nm, show visible absorption spectra of Zn-chlorine-e6 complex, Zn-chlorine-e6 complex immobilized on SHA and Zn-chlorine-e6 complex immobilized on PVP, all in water, respectively. The absorption spectra have main absorption peaks at λₘₐₓ=414 and 634 nm for Zn-chlorine-e6 complex, λₘₐₓ=418 and 636 nm for Zn-chlorine-e6 complex immobilized on SHA, and λₘₐₓ=416 and 638 nm for Zn-chlorine-e6 complex immobilized on PVP. The conclusions, drawn from these absorption spectra regarding the purity and stability of the monomeric products, were confirmed at every stage of the synthesis with the help of the highly sensitive analytical method of fluorescence spectroscopy (see FIGS. 9 to 14, discussed below).

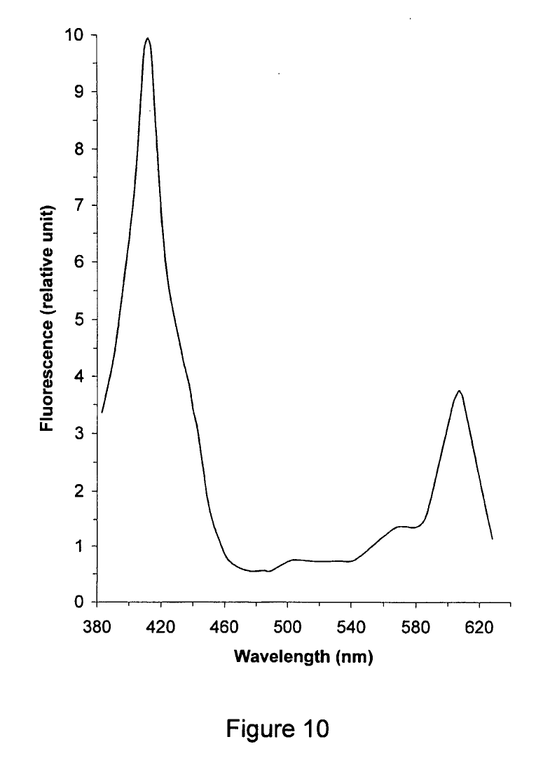

FIGS. 9 and 10 show the fluorescence spectrum and the fluorescence stimulation spectrum of Zn-chlorine-e6 complex in water respectively. The monomeric Zn-chlorine-e6 complex has a characteristic fluorescence spectrum with λₘₐₓ=643 nm, and a fluorescence stimulation spectrum with main peaks at λₘₐₓ=412 and 607 nm, i.e. analogous to the peaks observed in the absorption spectrum. This shows that the fluorescence belongs to the monomeric Zn-chlorine-e6 complex and the fluorescence data prove the high purity and homogeneity of the studied product.

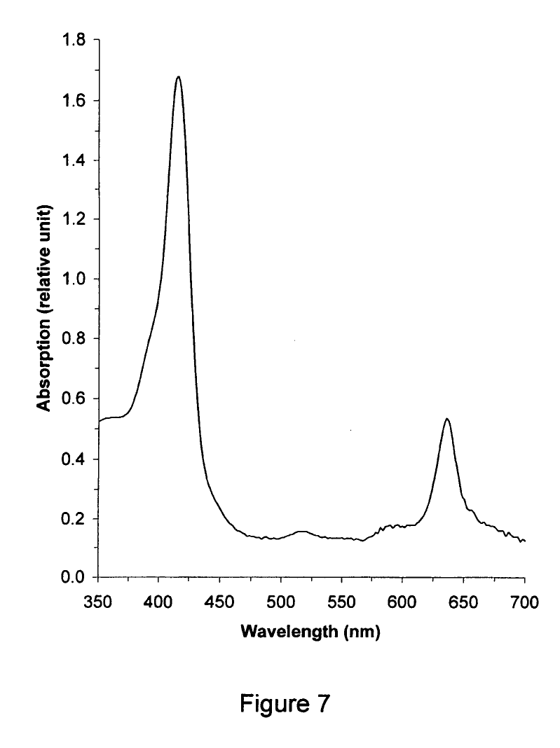

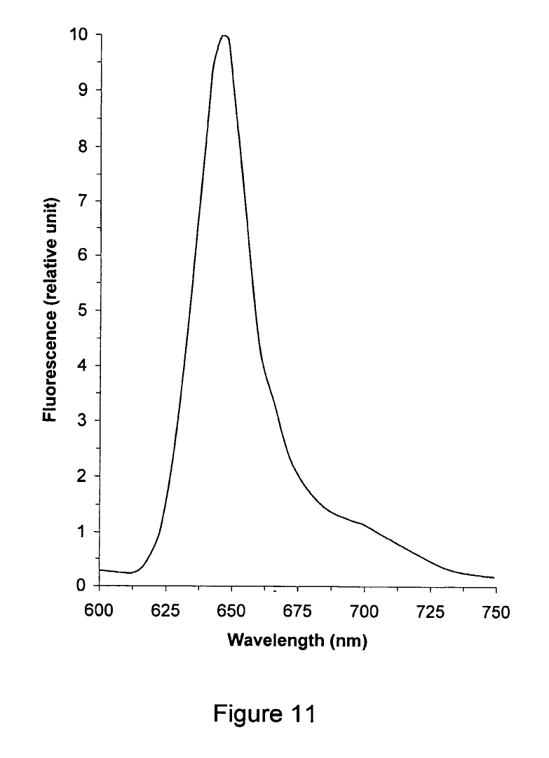

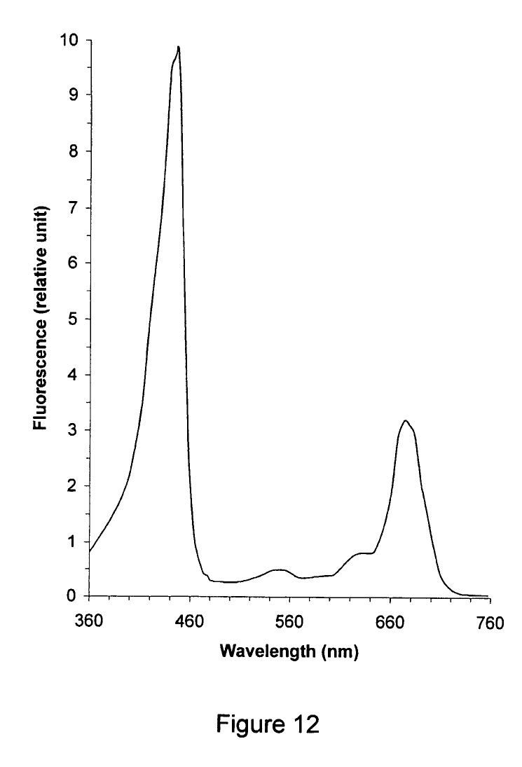

FIGS. 11 and 12 show the fluorescence spectrum and the fluorescence stimulation spectrum of Zn-chlorine-e6 complex immobilized on SHA in water respectively. The fluorescence spectrum is similar to the fluorescence spectrum of Zn-chlorine-e6 complex in water, though slightly shifted into the red region (λₘₐₓ=645 nm) and with peaks of a smaller half-width, which demonstrates the great structural similarity between the centres of Zn-chlorine-e6 complex and Zn-chlorine-e6 complex immobilized on SHA observed in these spectra. The fluorescence stimulation spectrum of Zn-chlorine-e6 complex immobilized on SHA, shown in FIG. 12, is very similar to its absorption spectrum shown in FIG. 7 and shows two main peaks at λₘₐₓ=446 and 673 nm with a smaller half-width and a more regular shape compared to the peaks in the absorption spectrum. This proves that the fluorescence belongs to monomeric Zn-chlorine-e6 complex immobilized on SHA and that the studied product has a high homogeneity and purity.

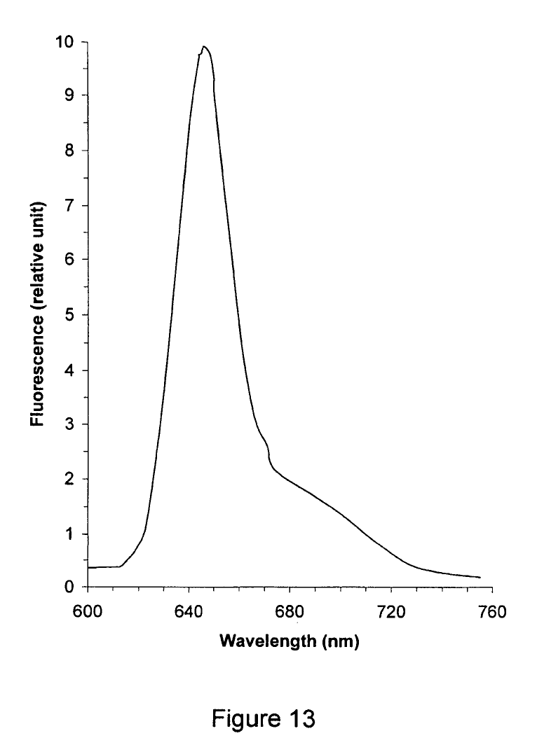

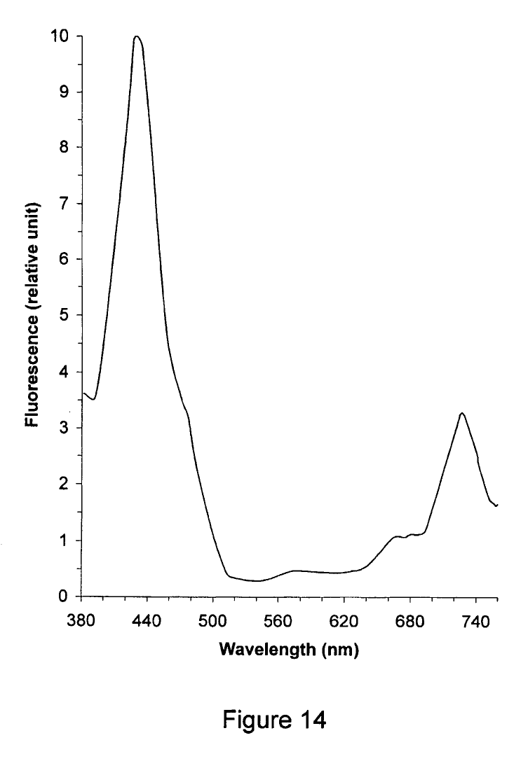

FIGS. 13 and 14 show the fluorescence spectrum and the fluorescence stimulation spectrum of Zn-chlorine-e6 complex immobilized on PVP in water respectively. The shape of the fluorescence spectrum is very similar to the fluorescence spectra discussed above and has a peak at λₘₐₓ=645 nm as in the spectrum of Zn-chlorine-e6 complex immobilized on SHA. The fluorescence stimulation spectrum has main peaks at λₘₐₓ=429 and 727 nm, which agrees with its absorption spectrum and shows that the fluorescence belongs to Zn-chlorine-e6 complex immobilized on PVP and that the product is highly pure.

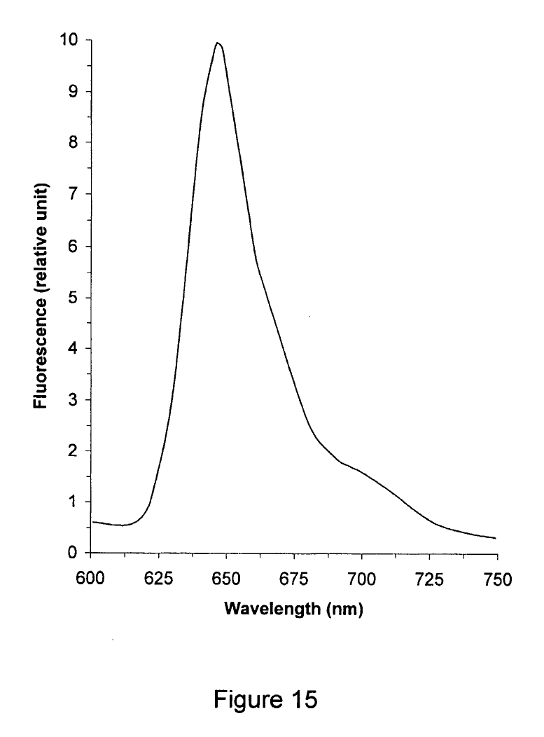

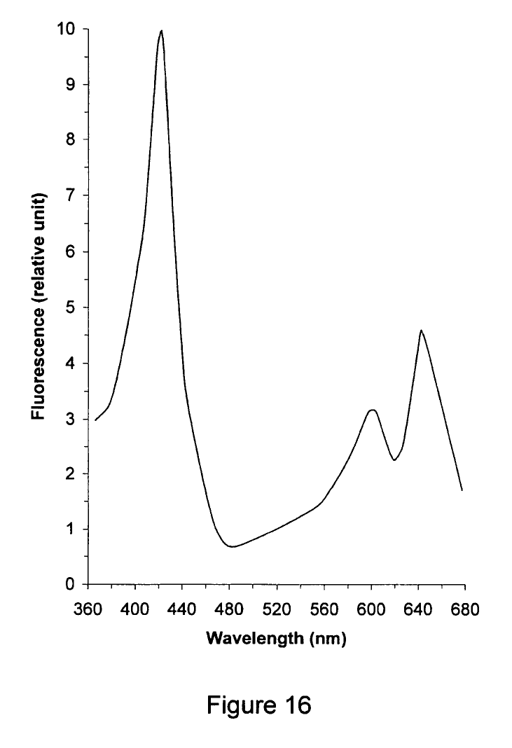

FIGS. 15 and 16 show the fluorescence spectrum and the fluorescence stimulation spectrum of a biological sample taken from the liquid above the sediment of an ascite turnout taken from an experimental animal (mouse), which had previously been injected intraabdominally with a preparation comprising Zn-chlorine-e6 complex immobilized on SHA. As can be seen by comparing the spectra of the biological sample shown in FIGS. 15 and 16 with the corresponding spectra of the models shown in FIGS. 9 to 14, the peaks in the spectra of the biological sample occur at similar λₘₐₓ (fluorescence spectrum in FIG. 15: λₘₐₓ=645 nm; fluorescence stimulation spectrum in FIG. 16: λₘₐₓ=418 and 641 nm) and have a similar peak shape and peak intensity ratio as the peaks in the spectra of the models. This means that the preparation injected into the experimental animal did not undergo substantial structural changes and comprises Zn-chlorine-e6 with a high structural homogeneity of the absorbing and fluorescent centre as was observed for Zn-chlorine-e6 complex immobilized on SHA.

### Example 5

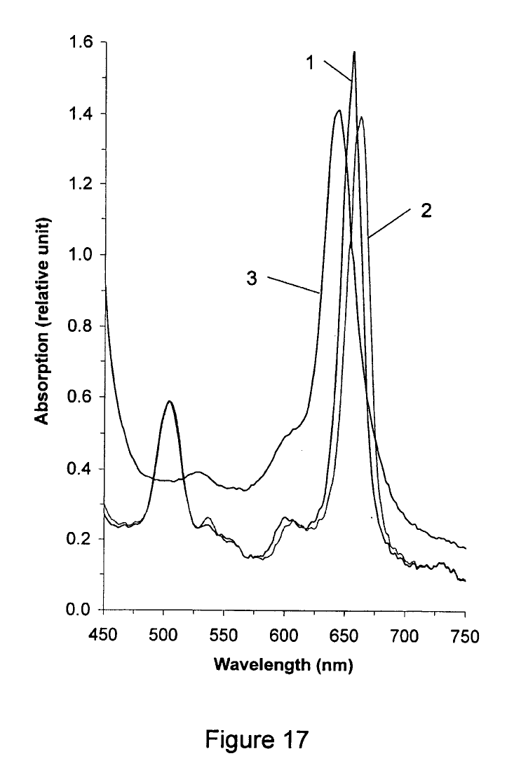

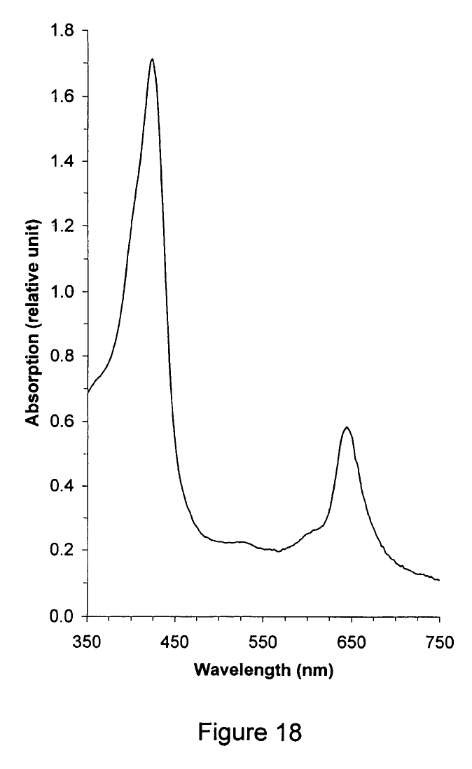

Cd-chlorine-e6 complex immobilized on PVP was synthesized in a similar way to Zn-chlorine-e6 complex immobilized on PVP (see Example 4). FIG. 17 shows the long-wave part of the visible absorption spectra of (1) the starting material chlorine-e6 (λₘₐₓ=656 nm), (2) chlorine-e6 immobilized on PVP (λₘₐₓ=662 nm), and (3) Cd-chlorine-e6 complex immobilized on PVP (λₘₐₓ=646 nm). FIG. 18 shows the absorption spectrum in the range of 350-750 nm of the monomer form of Cd-chlorine-e6 complex immobilized on PVP in water. As can be seen in FIG. 18, the spectrum of Cd-chlorine-e6 complex immobilized on PVP in monomer form has two main peaks at λₘₐₓ=424 and 646 nm respectively.

### Example 6

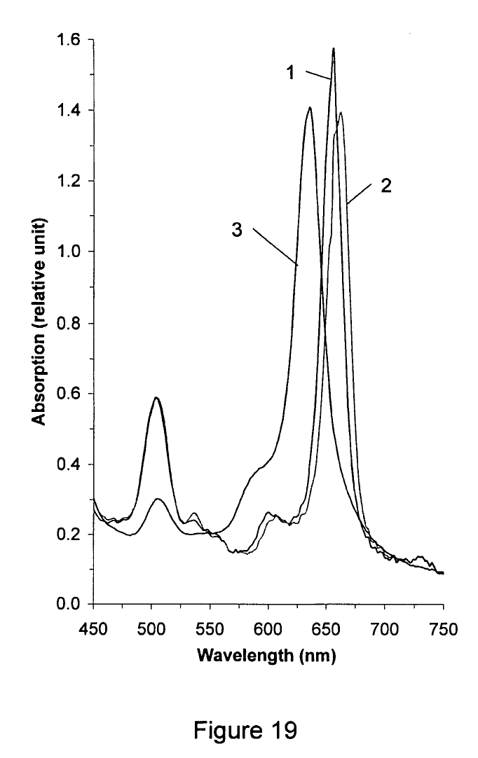

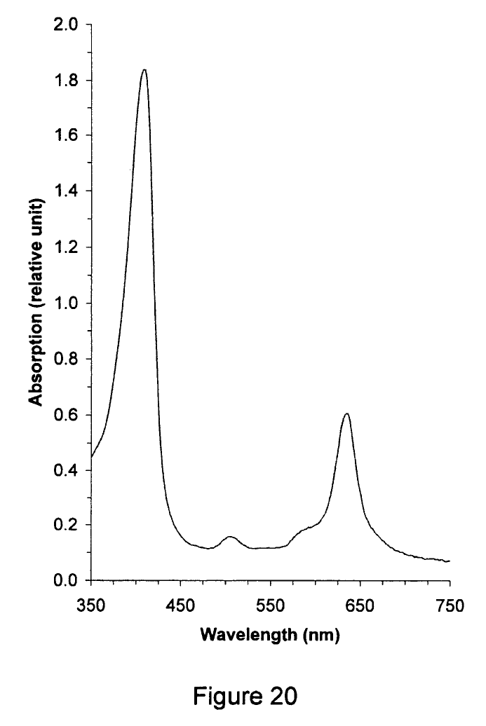

Cd-chlorine-e6 complex immobilized on PVP was synthesized in a similar way to Zn-chlorine-e6 complex immobilized on PVP (see Example 4). FIG. 19 shows the long-wave part of the visible absorption spectra of (1) the starting material chlorine-e6 (λₘₐₓ=656 nm), (2) chlorine-e6 immobilized on PVP (λₘₐₓ=662 nm), and (3) Cu-chlorine-e6 complex immobilized on PVP (λₘₐₓ=636 nm). FIG. 20 shows the absorption spectrum in the range of 350-750 nm of the monomer form of Cu-chlorine-e6 complex immobilized on PVP in water. As can be seen in FIG. 20, the absorption spectrum of Cu-chlorine-e6 complex immobilized on PVP in monomer form differs from the monomer spectra of Zn-chlorine-e6 immobilized complex and Cd-chlorine-e6 immobilized complex. The absorption spectrum of Cu-chlorine-e6 complex immobilized on PVP in monomer form has three main peaks at λₘₐₓ=410, 505 and 636 nm respectively.

### Preclinical Pharmacokinetic Studies

A remarkable and important feature of the immobilized monomer Zn-chlorine-e6 complex is the possibility of preparing a stable form of the monomeric Zn-chlorine-e6 complex at a pH of from 6 to 8.5, which is required for injection usage. Zn-chlorine-e6 complex preparations suitable for injection may be prepared by acidifying the reaction medium after completion of the synthesis. Pharmaceutically acceptable additives, which do not interfere with the structural stability of the Zn-chlorine-e6 complex and the homogeneity of the preparation, may be added to such preparations suitable for injection.

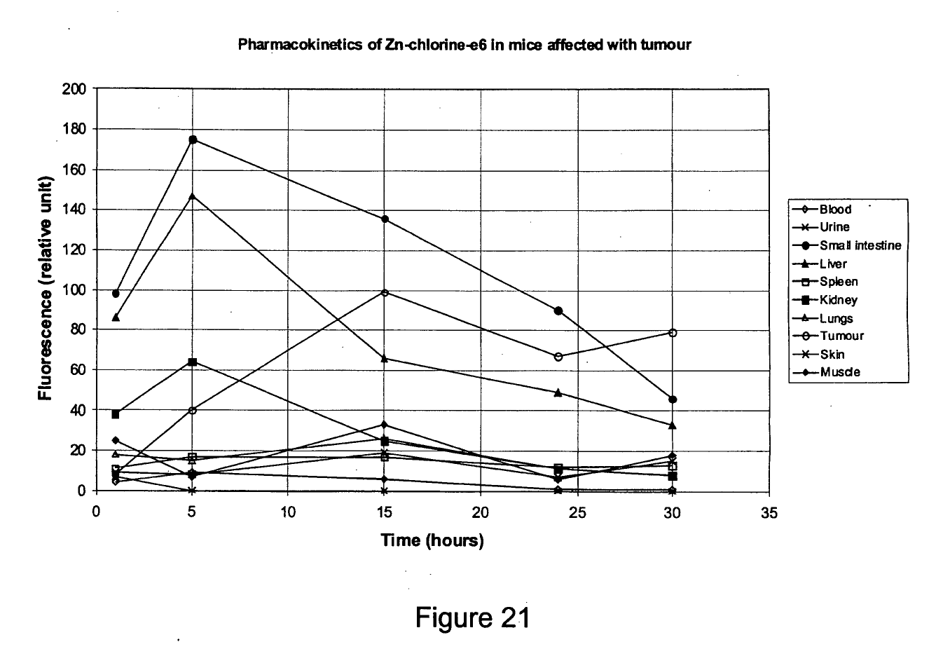

The pharmacokinetic distribution of Zn-chlorine-e6 complex immobilized on SHA over 30 hours in organs, tissues, biological liquids and tumours (embryocarcinoma) was studied. Female mice of the line Balb/c weighing 20-21 g were used as experimental animals. The pharmacokinetic studies were carried out using a Perkin-Elmer spectrofluorimeter on homogenates of organs and tumours, taken after the intraabdominal injection of Zn-chlorine-e6 complex immobilized on SHA at a dose of 25 mg/kg weight. The results of these pharmacokinetic distribution studies are depicted in FIG. 21 and summarised in Table 1 below.

TABLE 1

| | Amount of Zn-chlorine-e6 complex immobilized | | | | |
|---|---|---|---|---|---|
| | on SHA accumulated in organ X hours after | | | | |
| | injection in relative units | | | | |
| Organ | X = 1 | X = 5 | X = 15 | X = 24 | X = 30 |
| 1. Blood | 4 | 9 | 6 | 1 | 1 |
| 2. Urine | 7 | 0 | 0 | 0 | 0 |
| 3. Small intestine | 98 | 175 | 136 | 90 | 46 |
| 4. Liver | 86 | 147 | 66 | 49 | 33 |
| 5. Spleen | 11 | 17 | 17 | 12 | 13 |
| 6. Kidney | 38 | 64 | 25 | 11 | 8 |
| 7. Lungs | 18 | 15 | 26 | 11 | 8 |
| 8. Tumour | 8 | 40 | 99 | 67 | 79 |
| 9. Skin | 9 | 8 | 19 | 7 | 15 |
| 10. Muscle | 25 | 7 | 33 | 6 | 18 |

### Results:

A. Intraabdominal injection of Zn-chlorine-e6 complex immobilized on SHA at a dose of 25 mg/kg weight was well endured by the animals without any signs of toxicity and did not affect their behavioural reaction, both immediately and 30 hours after the injection.
B. The immobilized complex was rapidly absorbed from the abdominal cavity into the blood and was deposited in the liver during the first hours after injection. Its content in the liver tissues was 10-14 times higher than its level in the blood.
C. A significant quantity of the immobilized complex was also accumulated in the kidneys in the first 12 hours after injection (only 2-2.5 times less than in the liver), however, the immobilized complex was practically absent from the urine. During the next 18 hours, the immobilized complex was washed out intensely from the kidney tissue into the blood. The kidneys' secretion function was not affected during the whole observation period.
D. The maximum concentrations of the immobilized complex in the liver were found during the first 8 hours after injection. During the next 24 hours, the surplus of the immobilized complex was discharged intensely into the small intestines. The dynamics of the distribution curves of the liver and small intestines correlate precisely with one another. It may be sufficient to inject 5-10 times smaller doses of the immobilized complex in order to achieve maximum concentrations in the tumour.
E. 5-8 times less of the immobilized complex accumulated in the spleen and the lungs compared to the liver or tumour, and 24 hours after injection the spleen and lungs had phone readings.
F. Skin and muscle tissue both had practically the same accumulation dynamics, the only difference being that the immobilized complex content in the muscle was 1.5 times higher in the first 15 hours than the immobilized complex content in the skin.
G. The accumulation of the immobilized complex in turnout increased progressively from the moment of injection and reached its maximum 15 hours after injection. The maximum concentration plateau (12-20 hours) was found to be much longer than after chlorine-e6 injection, and after an insignificant fall by the end of the first 24 hours, a second increase of immobilized complex concentration up to the maximum readings of the concentration plateau was observed between 24 and 30 hours. A “scissors” effect (the immobilized complex concentration in the tumour is increasing, while the immobilized complex concentration in the liver is decreasing) was observed twice for the liver and tumour, once 12 hours after injection and once, even more pronounced, 24 hours after injection.

To summarise, after the absorption of the immobilized complex in the abdominal cavity, redistribution from the blood into the organs and washing out of the immobilized complex surplus by the liver during the first 24 hours, the immobilized complex accumulated in the tumour tissue in a concentration of 2.5 times greater than in the liver and 6 times greater than in the skin, muscle and other parenchymal organs. In comparison with the pharmacokinetics of the dimer form, the monomer form demonstrated much greater tumour selectivity and stability of the chemical structure in tissues.

The spectroscopic data (see FIGS. 1 to 20) and pharmacokinetic data (see FIG. 21) discussed above show that the immobilized preparations preserve the monomeric structure, purity and chemical stability of the porphyrin nucleus of the Zn-chlorine-e6 complex.

### Definition of Acute Toxicity Parameters:

To define parameters LD₁₀ and LD₅₀ three preparation doses were chosen (100, 125 and 150 mg/kg weight) for single intraabdominal injection. Chlorine-e6 readings were taken as a prototype, where LD₁₀ is 119 mg/kg weight and LD₅₀ is 160 mg/kg weight.

After injection of Zn-chlorine-e6 complex immobilized on SHA in the above stated doses, a first reaction to the injection was observed only with the third animal group (150 mg/kg weight), because the preparation was injected in 3 ml physiological solution, which caused temporal animal stillness due to abdominal swelling. After the absorption of the surplus liquid, however, these animals did not differ from the animals in the other two groups in their behavioural reactions (moving activity, defence reflex, food reflex, coat condition).

During the following 72 hours, signs of acute toxicity (slow reaction, hollow sides, diarrhoea, defence and food reflex absence) did not appear. The animals were kept under observation for a further fortnight.

Further tests were carried out similarly with intraabdominal injections of 175, 200 and 225 mg/kg weight, as well as 300, 350 and 450 mg/kg weight. None of these concentrations proved toxic.

It will be understood that the present invention has been described above by way of example only. The examples are not intended to limit the scope of the invention. Various modifications and embodiments can be made without departing from the scope of the invention, which is defined by the following claims.

1. Use of a compound for the manufacture of a medicament for the treatment of acne, Aids, viral hepatitis, diabetic retinopathy, infection with sars virus, coronary artery stenosis, carotid artery stenosis, intermittent claudication, Asian (chicken) flu virus, cervical dysplasia or cancer of the blood, cervix, naso-pharynx, trachea, larynx, bronchi, bronchioles, bladder, esophagus, stomach, rectum, colon, prostate, hollow organs, bile duct, ureter, kidney, uterus, vaginal or other female adnexa, wherein the compound is of formula 3 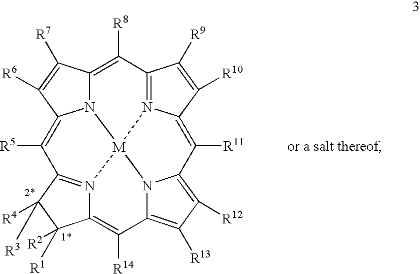
wherein
M is a metal atom in the M(II) oxidation state, a metal halide, a metal oxide or a silicon with two substituents,
each R¹, R², R³, R⁴, R⁵, R⁶, R⁷, R⁸, R⁹, R¹⁰, R¹¹, R¹², R¹³ and R¹⁴ is independently hydrogen, (CH₂)ₙ—CHO, (CH₂)ₙ—CO₂R¹⁵ or a C₁-C₆ saturated or unsaturated alkyl group optionally substituted with one or more of —OH and —NH₂,
n is 0, 1, 2 or 3, and
each R¹⁵ is independently hydrogen, lithium, sodium, potassium, magnesium, calcium, a C₁-C₆ saturated or unsaturated alkyl group optionally substituted with one or more of —OH and —NH₂, or a naturally occurring amino acid.

2. Use of a compound for the manufacture of a phototherapeutic agent for the use in photodynamic therapy or cytoluminescent therapy for the treatment of acne, Aids, viral hepatitis, diabetic retinopathy, infection with sars virus, coronary artery stenosis, carotid artery stenosis, intermittent claudication, Asian (chicken) flu virus, cervical dysplasia or cancer of the blood, cervix, naso-pharynx, trachea, larynx, bronchi, bronchioles, bladder, esophagus, stomach, rectum, colon, prostate, hollow organs, bile duct, ureter, kidney, uterus, vaginal or other female adnexa, wherein the compound is of formula 3 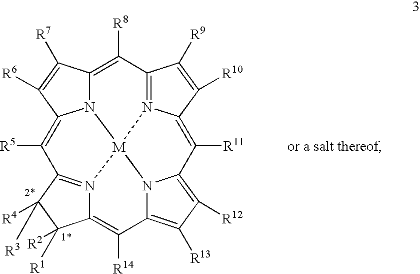
wherein
M is a metal atom in the M(II) oxidation state, a metal halide, a metal oxide or a silicon with two substituents,
each R¹, R², R³, R⁴, R⁵, R⁶, R⁷, R⁸, R⁹, R¹⁰, R¹¹, R¹², R¹³ and R¹⁴ is independently hydrogen, (CH₂)ₙ—CHO, (CH₂)—CO₂R¹⁵ or a C₁-C₆ saturated or unsaturated alkyl group optionally substituted with one or more of —OH and —NH₂,
n is 0, 1, 2 or 3, and
each R¹⁵ is independently hydrogen, lithium, sodium, potassium, magnesium, calcium, a C₁-C₆ saturated or unsaturated alkyl group optionally substituted with one or more of —OH and —NH₂, or a naturally occurring amino acid.

3. Use of a compound for the manufacture of a photodiagnostic agent for the detection of acne, Aids, viral hepatitis, diabetic retinopathy, infection with sars virus, coronary artery stenosis, carotid artery stenosis, intermittent claudication, Asian (chicken) flu virus, cervical dysplasia, or cancer of the blood, cervix, naso-pharynx, trachea, larynx, bronchi, bronchioles, bladder, esophagus, stomach, rectum, colon, prostate, hollow organs, bile duct, ureter, kidney, uterus, vaginal or other female adnexa, or atherosclerosis, multiple sclerosis, diabetes, arthritis, rheumatoid arthritis, a fungal, viral, chlamydial, bacterial, nanobacterial or parasitic infectious disease, HIV, hepatitis, herpes simplex, herpes zoster, psoriasis, a cardiovascular disease, or a dermatological condition, wherein the compound is of formula 3 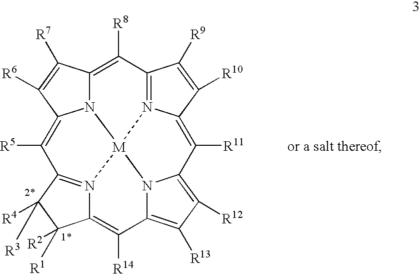
wherein
M is a metal atom in the M(II) oxidation state, a metal halide, a metal oxide or a silicon with two substituents,
each R¹, R², R³, R⁴, R⁵, R⁶, R⁷, R⁸, R⁹, R¹⁰, R¹¹, R¹², R¹³ and R¹⁴ is independently hydrogen, (CH₂)₂—CHO, (CH₂)ₙ—CO₂R¹⁵ or a C₁-C₆ saturated or unsaturated alkyl group optionally substituted with one or more of —OH and —NH₂,
n is 0, 1, 2 or 3, and
each R¹⁵ is independently hydrogen, lithium, sodium, potassium, magnesium, calcium, a C₁-C₆ saturated or unsaturated alkyl group optionally substituted with one or more of —OH and —NH₂, or a naturally occurring amino acid.

4. A use as claimed in claim 3, wherein the photodiagnostic agent is for the fluorescent or phosphorescent detection of the said diseases.

5. A use as claimed in claim 3 or claim 4, wherein the photodiagnostic agent is for the fluorescent or phosphorescent detection and quantification of the said diseases.

6. A use as claimed in any one of claims 3 to 5, wherein the photodiagnostic agent is for the detection of acne, Aids, viral hepatitis, diabetic retinopathy, infection with sars virus, coronary artery stenosis, carotid artery stenosis, intermittent claudication, Asian (chicken) flu virus, cervical dysplasia, or cancer of the blood, cervix, naso-pharynx, trachea, larynx, bronchi, bronchioles, bladder, esophagus, stomach, rectum, colon, prostate, hollow organs, bile duct, ureter, kidney, uterus, vaginal or other female adnexa.

7. A use as claimed in any one of claims 1 to 6, wherein the medicament, phototherapeutic agent or photodiagnostic agent is for the treatment or detection of acne, Aids, viral hepatitis, diabetic retinopathy, infection with sars virus, coronary artery stenosis, carotid artery stenosis, intermittent claudication, or Asian (chicken) flu virus.

8. A use as claimed in any one of claims 1 to 6, wherein the medicament, phototherapeutic agent or photodiagnostic agent is for the treatment or detection of early cancer.

9. A use as claimed in any one of claims 1 to 8, wherein the medicament, phototherapeutic agent or photodiagnostic agent is adapted for administration simultaneous with or prior to administration of irradiation or sound.

10. A use as claimed in claim 9, wherein the medicament, phototherapeutic agent or photodiagnostic agent is adapted for administration prior to administration of irradiation.

11. A use as claimed in claim 10, wherein the medicament or phototherapeutic agent is adapted for administration 10 to 100 hours before the irradiation.

12. A use as claimed in claim 11, wherein the medicament or phototherapeutic agent is adapted for administration 50 to 90 hours before the irradiation.

13. A use as claimed in claim 12, wherein the medicament or phototherapeutic agent is adapted for administration about 72 hours before the irradiation.

14. A use as claimed in claim 10, wherein the photodiagnostic agent is adapted for administration 3 to 60 hours before the irradiation.

15. A use as claimed in claim 14, wherein the photodiagnostic agent is adapted for administration 8 to 40 hours before the irradiation.

16. A use as claimed in any one of claims 9 to 15, wherein the irradiation is electromagnetic radiation with a wavelength in the range of from 500 nm to 1000 nm.

17. A use as claimed in claim 16, wherein the irradiation is electromagnetic radiation with a wavelength in the range of from 600 nm to 900 nm.

18. A use as claimed in claim 17, wherein the irradiation is electromagnetic radiation with a wavelength in the range of from 620 nm to 820 nm.

19. A use as claimed in claim 18, wherein the irradiation is electromagnetic radiation with a wavelength in the range of from 630 nm to 710 nm.

20. A use as claimed in any one of the preceding claims, wherein M is Ca, Ti, V, Nb, Cr, Mo, Mn, Tc, Ru, Co, Rh, Ni, Pd, Pt, Ag, Au, Zn, Cd, Hg, Al, Ga, In, Ge, Pb, a lanthanide, or SiR₂ where R is a C₁-C₈ saturated or unsaturated alkyl group.

21. A use as claimed in any one of the preceding claims, wherein the compound is immobilized on a protein, a polypeptide, a polymer or activated charcoal.

22. A use as claimed in any one of the preceding claims, wherein the compound is immobilized in monomer form.

23. A use as claimed in claim 21 or claim 22, wherein the compound is immobilized on serum humane albumin (SHA), bovine serum albumin (BSA) or polyvinylpyrrolidone (PVP).

24. A use as claimed in claim 21 or claim 22, wherein the compound is immobilized on a low molecular weight polypeptide.

25. A use as claimed in claim 24, wherein the compound is immobilized on polylysine or polyasparagine.

26. A use as claimed in any one of the preceding claims, wherein the compound is linked to a photosensitive material.

27. A use as claimed in claim 26, wherein the photosensitive material is a nano-dot.

28. A use as claimed in any one of the preceding claims, wherein M is Mg, Ca, Ti, V, Nb, Cr, Mo, Mn, Tc, Fe, Ru, Co, Rh, Ni, Pd, Pt, Cu, Ag, Au, Zn, Cd, Hg, Al, Ga, In, Ge, Sn, Pb, a lanthanide, or SiR₂ where R is a C₁-C₈ saturated or unsaturated alkyl group.

29. A use as claimed in claim 28, wherein M is Mg, Ca, Ti, V, Nb, Cr, Mo, Mn, Tc, Fe, Ru, Co, Rh, Ni, Pd, Pt, Cu, Ag, Au, Zn, Cd, Hg, Al, Ga, In, Ge, Sn, Pb or a lanthanide.

30. A use as claimed in claim 29, wherein M is Zn, Cu, Cd, Ca, Mn, Au or Co.

31. A use as claimed in claim 20 or claim 30, wherein M is Zn, Cd, Ca, Mn, Au or Co.

32. A use as claimed in claim 31, wherein M is Zn.

33. A use as claimed in any one of the preceding claims, wherein each R¹, R², R³, R⁴, R⁵, R⁶, R⁷, R⁸, R⁹, R¹⁰, R¹¹, R¹², R¹³ and R¹⁴ is independently hydrogen, methyl, ethyl, propyl, allyl, CO₂H, CH₂CO₂H or (CH₂)₂CO₂H.

34. A use as claimed in any one of the preceding claims, wherein R¹ and R³ are hydrogen.

35. A use as claimed in any one of the preceding claims, wherein R⁵, R⁸ and R¹¹ are hydrogen.

36. A use as claimed in any one of the preceding claims, wherein R¹⁵ is hydrogen, sodium, a C₁-C₆ saturated or unsaturated alkyl group or a naturally occurring amino acid.

37. A use as claimed in any one of the preceding claims, wherein R¹⁵ is aspartic acid or lysine.

38. A use as claimed in any one of the preceding claims, wherein the compound is substantially enantiomerically pure.

39. A use as claimed in any one of the preceding claims, wherein R¹ and R³ are hydrogen, and R¹ is in the down-configuration and R³ is in the up-configuration in formula 3 as shown.

40. A use as claimed in any one of the preceding claims, wherein R¹ and R³ are hydrogen, R² is (CH₂)₂CO₂H, R⁴ is CO₂H, and chiral centres 1* and 2* are in the (S)-configuration.

41. A use as claimed in any one of the preceding claims, wherein the compound is of formula 2

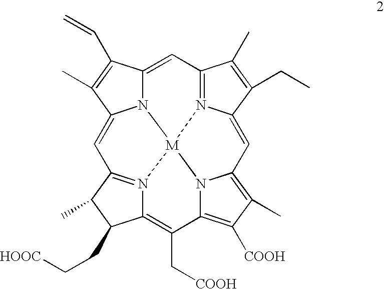

42. A method of treating acne, Aids, viral hepatitis, diabetic retinopathy, infection with sars virus, coronary artery stenosis, carotid artery stenosis, intermittent claudication, Asian (chicken) flu virus, cervical dysplasia or cancer of the blood, cervix, naso-pharynx, trachea, larynx, bronchi, bronchioles, bladder, esophagus, stomach, rectum, colon, prostate, hollow organs, bile duct, ureter, kidney, uterus, vaginal or other female adnexa, comprising administering a therapeutically effective amount of a compound to a human or animal in need thereof, wherein the compound is of formula 3 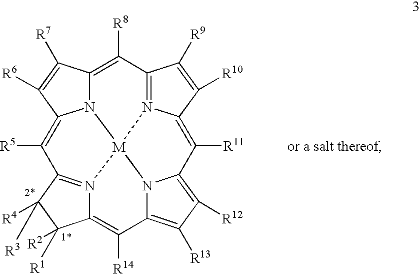
wherein
M is a metal atom in the M(II) oxidation state, a metal halide, a metal oxide or a silicon with two substituents,
each R¹, R², R³, R⁴, R⁵, R⁶, R⁷, R⁸, R⁹, R¹⁰, R¹¹, R¹², R¹³ and R¹⁴ is independently hydrogen, (CH₂)ₙ—CHO, (CH₂)ₙ—CO₂R¹⁵ or a C₁-C₆ saturated or unsaturated alkyl group optionally substituted with one or more of —OH and —NH₂,
n is 0, 1, 2 or 3, and
each R¹⁵ is independently hydrogen, lithium, sodium, potassium, magnesium, calcium, a C₁-C₆ saturated or unsaturated alkyl group optionally substituted with one or more of —OH and —NH₂, or a naturally occurring amino acid.

43. A method as claimed in claim 42, wherein the human or animal is further subjected to irradiation or sound.

44. A method of photodynamic therapy or cytoluminescent therapy of a human or animal disease, comprising administering a therapeutically effective amount of a compound to a human or animal in need thereof and subjecting the human or animal to irradiation or sound, wherein the human or animal disease is acne, Aids, viral hepatitis, diabetic retinopathy, infection with sars virus, coronary artery stenosis, carotid artery stenosis, intermittent claudication, Asian (chicken) flu virus, cervical dysplasia or cancer of the blood, cervix, naso-pharynx, trachea, larynx, bronchi, bronchioles, bladder, esophagus, stomach, rectum, colon, prostate, hollow organs, bile duct, ureter, kidney, uterus, vaginal or other female adnexa, and wherein the compound is of formula 3 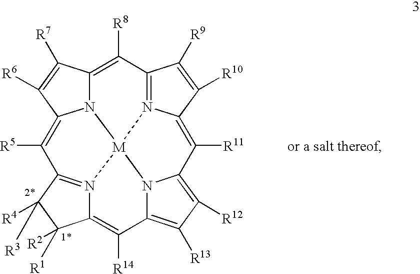
wherein
M is a metal atom in the M(II) oxidation state, a metal halide, a metal oxide or a silicon with two substituents,
each R¹, R², R³, R⁴, R⁵, R⁶, R⁷, R⁸, R⁹, R¹⁰, R¹¹, R¹², R¹³ and R¹⁴ is independently hydrogen, (CH₂)ₙ—CHO, (CH₂)ₙ—CO₂R¹⁵ or a C₁-C₆ saturated or unsaturated alkyl group optionally substituted with one or more of —OH and —NH₂,
n is 0, 1, 2 or 3, and
each R¹⁵ is independently hydrogen, lithium, sodium, potassium, magnesium, calcium, a C₁-C₆ saturated or unsaturated alkyl group optionally substituted with one or more of —OH and —NH₂, or a naturally occurring amino acid.

45. A method as claimed in claim 43 or claim 44, wherein the human or animal is subjected to irradiation or sound simultaneously with or after administration of the compound of formula 3 or a salt thereof.

46. A method as claimed in claim 45, wherein the human or animal is subjected to irradiation after administration of the compound of formula 3 or a salt thereof.

47. A method as claimed in claim 46, wherein the human or animal is subjected to irradiation 10 to 100 hours after administration of the compound of formula 3 or a salt thereof.

48. A method as claimed in claim 47, wherein the human or animal is subjected to irradiation 50 to 90 hours after administration of the compound of formula 3 or a salt thereof.

49. A method as claimed in claim 48, wherein the human or animal is subjected to irradiation about 72 hours after administration of the compound of formula 3 or a salt thereof.

50. A method of photodiagnosis for the detection of acne, Aids, viral hepatitis, diabetic retinopathy, infection with sars virus, coronary artery stenosis, carotid artery stenosis, intermittent claudication, Asian (chicken) flu virus, cervical dysplasia, or cancer of the blood, cervix, naso-pharynx, trachea, larynx, bronchi, bronchioles, bladder, esophagus, stomach, rectum, colon, prostate, hollow organs, bile duct, ureter, kidney, uterus, vaginal or other female adnexa, or atherosclerosis, multiple sclerosis, diabetes, arthritis, rheumatoid arthritis, a fungal, viral, chlamydial, bacterial, nanobacterial or parasitic infectious disease, HIV, hepatitis, herpes simplex, herpes zoster, psoriasis, a cardiovascular disease, or a dermatological condition, in a human or animal, comprising administering a compound to a human or animal and subjecting the human or animal to irradiation or sound, wherein the compound is of formula 3 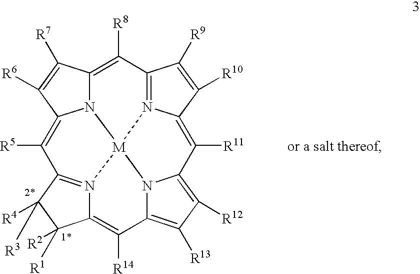
wherein
M is a metal atom in the M(II) oxidation state, a metal halide, a metal oxide or a silicon with two substituents,
each R¹, R², R³, R⁴, R⁵, R⁶, R⁷, R⁸, R⁹, R¹⁰, R¹¹, R¹², R¹³ and R¹⁴ is independently hydrogen, (CH₂)ₙ—CHO, (CH₂)₂—CO₂R¹⁵ or a C₁-C₆ saturated or unsaturated alkyl group optionally substituted with one or more of —OH and —NH₂,
n is 0, 1, 2 or 3, and
each R¹⁵ is independently hydrogen, lithium, sodium, potassium, magnesium, calcium, a C₁-C₆ saturated or unsaturated alkyl group optionally substituted with one or more of —OH and —NH₂, or a naturally occurring amino acid.

51. A method as claimed in claim 50, wherein the photodiagnosis is for the fluorescent or phosphorescent detection of the said diseases.

52. A method as claimed in claim 50 or claim 51, wherein the photodiagnosis is for the fluorescent or phosphorescent detection and quantification of the said diseases.

53. A method as claimed in any one of claims 50 to 52, wherein the human or animal is subjected to irradiation or sound simultaneously with or after administration of the compound of formula 3 or a salt thereof.

54. A method as claimed in claim 53, wherein the human or animal is subjected to irradiation after administration of the compound of formula 3 or a salt thereof.

55. A method as claimed in claim 54, wherein the human or animal is subjected to irradiation 3 to 60 hours after administration of the compound of formula 3 or a salt thereof.

56. A method as claimed in claim 55, wherein the human or animal is subjected to irradiation 8 to 40 hours after administration of the compound of formula 3 or a salt thereof.

57. A method as claimed in any one of claims 50 to 56, wherein the method of photodiagnosis is for the detection of acne, Aids, viral hepatitis, diabetic retinopathy, infection with sars virus, coronary artery stenosis, carotid artery stenosis, intermittent claudication, Asian (chicken) flu virus, cervical dysplasia, or cancer of the blood, cervix, naso-pharynx, trachea, larynx, bronchi, bronchioles, bladder, esophagus, stomach, rectum, colon, prostate, hollow organs, bile duct, ureter, kidney, uterus, vaginal or other female adnexa.

58. A method as claimed in any one of claims 43 to 57, wherein the irradiation is electromagnetic radiation with a wavelength in the range of from 500 nm to 1000 nm.

59. A method as claimed in claim 58, wherein the irradiation is electromagnetic radiation with a wavelength in the range of from 600 nm to 900 nm.

60. A method as claimed in claim 59, wherein the irradiation is electromagnetic radiation with a wavelength in the range of from 620 nm to 820 nm.

61. A method as claimed in claim 60, wherein the irradiation is electromagnetic radiation with a wavelength in the range of from 630 nm to 710 nm.

62. A method as claimed in any one of claims 42 to 61, wherein the method of treatment, therapy or photodiagnosis is for the treatment or detection of acne, Aids, viral hepatitis, diabetic retinopathy, infection with sars virus, coronary artery stenosis, carotid artery stenosis, intermittent claudication, or Asian (chicken) flu virus.

63. A method as claimed in any one of claims 42 to 61, wherein the method of treatment, therapy or photodiagnosis is for the treatment or detection of early cancer.

64. A method as claimed in any one of claims 42 to 63, wherein M is Ca, Ti, V, Nb, Cr, Mo, Mn, Tc, Ru, Co, Rh, Ni, Pd, Pt, Ag, Au, Zn, Cd, Hg, Al, Ga, In, Ge, Pb, a lanthanide, or SiR₂ where R is a C₁-C₈ saturated or unsaturated alkyl group.

65. A method as claimed in any one of claims 42 to 64, wherein the compound is immobilized on a protein, a polypeptide, a polymer or activated charcoal.

66. A method as claimed in any one of claims 42 to 65, wherein the compound is immobilized in monomer form.

67. A method as claimed in claim 65 or claim 66, wherein the compound is immobilized on serum humane albumin (SHA), bovine serum albumin (BSA) or polyvinylpyrrolidone (PVP).

68. A method as claimed in claim 65 or claim 66, wherein the compound is immobilized on a low molecular weight polypeptide.

69. A method as claimed in claim 68, wherein the compound is immobilized on polylysine or polyasparagine.

70. A method as claimed in any one of claims 42 to 69, wherein the compound is linked to a photosensitive material.

71. A method as claimed in claim 70, wherein the photosensitive material is a nano-dot.

72. A method as claimed in any one of claims 42 to 71, wherein M is Mg, Ca, Ti, V, Nb, Cr, Mo, Mn, Tc, Fe, Ru, Co, Rh, Ni, Pd, Pt, Cu, Ag, Au, Zn, Cd, Hg, Al, Ga, In, Ge, Sn, Pb, a lanthanide, or SiR₂ where R is a C₁-C₈ saturated or unsaturated alkyl group.

73. A method as claimed in claim 72, wherein M is Mg, Ca, Ti, V, Nb, Cr, Mo, Mn, Tc, Fe, Ru, Co, Rh, Ni, Pd, Pt, Cu, Ag, Au, Zn, Cd, Hg, Al, Ga, In, Ge, Sn, Pb or a lanthanide.

74. A method as claimed in claim 73, wherein M is Zn, Cu, Cd, Ca, Mn, Au or Co.

75. A method as claimed in claim 64 or claim 74, wherein M is Zn, Cd, Ca, Mn, Au or Co.

76. A method as claimed in claim 75, wherein M is Zn.

77. A method as claimed in any one of claims 42 to 76, wherein each R¹, R², R³, R⁴, R⁵, R⁶, R⁷, R⁸, R⁹, R¹⁰, R¹¹, R¹², R¹³ and R¹⁴ is independently hydrogen, methyl, ethyl, propyl, allyl, CO₂H, CH₂CO₂H or (CH₂)₂CO₂H.

78. A method as claimed in any one of claims 42 to 77, wherein R¹ and R³ are hydrogen.

79. A method as claimed in any one of claims 42 to 78, wherein R⁵, R⁸ and R¹⁵ are hydrogen.

80. A method as claimed in any one of claims 42 to 79, wherein R¹⁵ is hydrogen, sodium, a C₁-C₆ saturated or unsaturated alkyl group or a naturally occurring amino acid.

81. A method as claimed in any one of claims 42 to 80, wherein R¹⁵ is aspartic acid or lysine.

82. A method as claimed in any one of claims 42 to 81, wherein the compound is substantially enantiomerically pure.

83. A method as claimed in any one of claims 42 to 82, wherein R¹ and R³ are hydrogen, and R¹ is in the down-configuration and R³ is in the up-configuration in formula 3 as shown.

84. A method as claimed in any one of claims 42 to 83, wherein R¹ and R³ are hydrogen, R² is (CH₂)₂CO₂H, R⁴ is CO₂H, and chiral centres 1* and 2* are in the (S)-configuration.

85. A method as claimed in any one of claims 42 to 84, wherein the compound is of formula 2

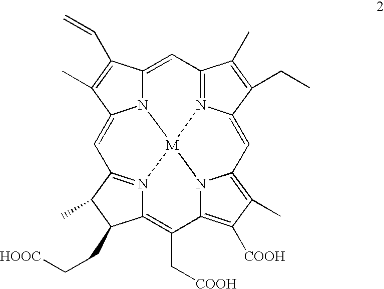

86. A method of cold sterilising a surgical or other device, comprising the steps of: providing a compound on the device and subjecting the device to irradiation or sound, wherein the compound is of formula 3 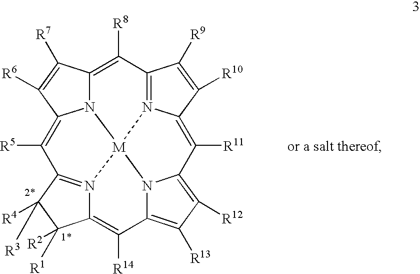
wherein
M is a metal atom in the M(II) oxidation state, a metal halide, a metal oxide or a silicon with two substituents,
each R¹, R², R³, R⁴, R⁵, R⁶, R⁷, R⁸, R⁹, R¹⁰, R¹¹, R¹², R¹³ and R¹⁴ is independently hydrogen, (CH₂)ₙ—CHO, (CH₂)—CO₂R¹⁵ or a C₁-C₆ saturated or unsaturated alkyl group optionally substituted with one or more of —OH and —NH₂,
n is 0, 1, 2 or 3, and
each R¹⁵ is independently hydrogen, lithium, sodium, potassium, magnesium, calcium, a C₁-C₆ saturated or unsaturated alkyl group optionally substituted with one or more of —OH and —NH₂, or a naturally occurring amino acid.

87. A method as claimed in claim 86, wherein the device is subjected to irradiation or sound simultaneously with or after provision of the compound of formula 3 or a salt thereof on the device.

88. A method as claimed in claim 87, wherein the device is subjected to irradiation after provision of the compound of formula 3 or a salt thereof on the device.

89. A method as claimed in any one of claims 86 to 88, wherein the irradiation is electromagnetic radiation with a wavelength in the range of from 500 nm to 1000 nm.

90. A method as claimed in claim 89, wherein the irradiation is electromagnetic radiation with a wavelength in the range of from 600 nm to 900 nm.

91. A method as claimed in claim 90, wherein the irradiation is electromagnetic radiation with a wavelength in the range of from 620 nm to 820 nm.

92. A method as claimed in claim 91, wherein the irradiation is electromagnetic radiation with a wavelength in the range of from 630 nm to 710 nm.

93. A method as claimed in any one of claims 86 to 92, wherein M is Ca, Ti, V, Nb, Cr, Mo, Mn, Tc, Ru, Co, Rh, Ni, Pd, Pt, Ag, Au, Zn, Cd, Hg, Al, Ga, In, Ge, Pb, a lanthanide, or SiR₂ where R is a C₁-C₈ saturated or unsaturated alkyl group.

94. A method as claimed in any one of claims 86 to 93, wherein the compound is immobilized on a protein, a polypeptide, a polymer or activated charcoal.

95. A method as claimed in any one of claims 86 to 94, wherein the compound is immobilized in monomer form.

96. A method as claimed in claim 94 or claim 95, wherein the compound is immobilized on serum humane albumin (SHA), bovine serum albumin (BSA) or polyvinylpyrrolidone (PVP).

97. A method as claimed in claim 94 or claim 95, wherein the compound is immobilized on a low molecular weight polypeptide.

98. A method as claimed in claim 97, wherein the compound is immobilized on polylysine or polyasparagine.

99. A method as claimed in any one of claims 86 to 98, wherein M is Mg, Ca, Ti, V, Nb, Cr, Mo, Mn, Tc, Fe, Ru, Co, Rh, Ni, Pd, Pt, Cu, Ag, Au, Zn, Cd, Hg, Al, Ga, In, Ge, Sn, Pb, a lanthanide, or SiR₂ where R is a C₁-C₈ saturated or unsaturated alkyl group.

100. A method as claimed in claim 99, wherein M is Mg, Ca, Ti, V, Nb, Cr, Mo, Mn, Tc, Fe, Ru, Co, Rh, Ni, Pd, Pt, Cu, Ag, Au, Zn, Cd, Hg, Al, Ga, In, Ge, Sn, Pb or a lanthanide.

101. A method as claimed in claim 100, wherein M is Zn, Cu, Cd, Ca, Mn, Au or Co.

102. A method as claimed in claim 93 or claim 101, wherein M is Zn, Cd, Ca, Mn, Au or Co.

103. A method as claimed in claim 102, wherein M is Zn.

104. A method as claimed in any one of claims 86 to 103, wherein each R¹, R², R³, R⁴, R⁵, R⁶, R⁷, R⁸, R⁹, R¹⁰, R¹¹, R¹², R¹³ and R¹⁴ is independently hydrogen, methyl, ethyl, propyl, allyl, CO₂H, CH₂CO₂H or (CH₂)₂CO₂H.

105. A method as claimed in any one of claims 86 to 104, wherein R¹ and R³ are hydrogen.

106. A method as claimed in any one of claims 86 to 105, wherein R⁵, R⁸ and R¹¹ are hydrogen.

107. A method as claimed in any one of claims 86 to 106, wherein R¹⁵ is hydrogen, sodium, a C₁-C₆ saturated or unsaturated alkyl group or a naturally occurring amino acid.

108. A method as claimed in any one of claims 86 to 107, wherein R¹⁵ is aspartic acid or lysine.

109. A method as claimed in any one of claims 86 to 108, wherein the compound is substantially enantiomerically pure.

110. A method as claimed in any one of claims 86 to 109, wherein R¹ and R³ are hydrogen, and R¹ is in the down-configuration and R³ is in the up-configuration in formula 3 as shown.

111. A method as claimed in any one of claims 86 to 110, wherein R¹ and R³ are hydrogen, R² is (CH₂)₂CO₂H, R⁴ is CO₂H, and chiral centres 1* and 2* are in the (S)-configuration.

112. A method as claimed in any one of claims 86 to 111, wherein the compound is of formula 2

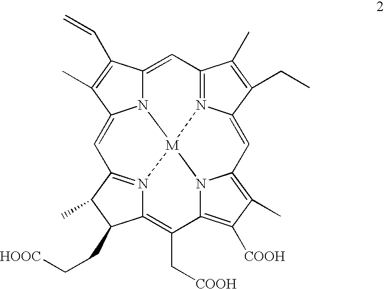

113. A compound of formula 3 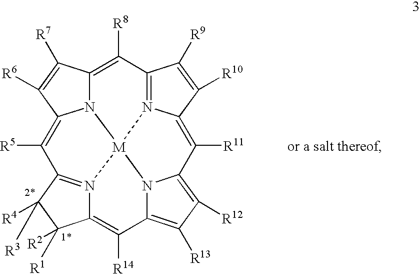
wherein
M is a metal atom in the M(II) oxidation state, a metal halide, a metal oxide or a silicon with two substituents,
each R¹, R², R³, R⁴, R⁵, R⁶, R⁷, R⁸, R⁹, R¹⁰, R¹¹, R¹², R¹³ and R¹⁴ is independently hydrogen, (CH₂)ₙ—CHO, (CH₂)ₙ—CO₂R¹⁵ or a C₁-C₆ saturated or unsaturated alkyl group optionally substituted with one or more of —OH and —NH₂,
n is 0, 1, 2 or 3, and
each R¹⁵ is independently hydrogen, lithium, sodium, potassium, magnesium, calcium, a C₁-C₆ saturated or unsaturated alkyl group optionally substituted with one or more of —OH and —NH₂, or a naturally occurring amino acid,
wherein the compound is linked or attached to a magnetic element.

114. A compound as claimed in claim 113, wherein the magnetic element is Gd, Fe or Mn.

115. A compound as claimed in claim 113 or claim 114, wherein M is Ca, Ti, V, Nb, Cr, Mo, Mn, Tc, Ru, Co, Rh, Ni, Pd, Pt, Ag, Au, Zn, Cd, Hg, Al, Ga, In, Ge, Pb, a lanthanide, or SiR₂ where R is a C₁-C₈ saturated or unsaturated alkyl group.

116. A compound as claimed in any one of claims 113 to 115, wherein the compound is immobilized on a protein, a polypeptide, a polymer or activated charcoal.

117. A compound as claimed in any one of claims 113 to 116, wherein the compound is immobilized in monomer form.

118. A compound as claimed in claim 116 or claim 117, wherein the compound is immobilized on serum humane albumin (SHA), bovine serum albumin (BSA) or polyvinylpyrrolidone (PVP).

119. A compound as claimed in claim 116 or claim 117, wherein the compound is immobilized on a low molecular weight polypeptide.

120. A compound as claimed in claim 119, wherein the compound is immobilized on polylysine or polyasparagine.

121. A compound as claimed, in any one of claims 113 to 120, wherein M is Mg, Ca, Ti, V, Nb, Cr, Mo, Mn, Tc, Fe, Ru, Co, Rh, Ni, Pd, Pt, Cu, Ag, Au, Zn, Cd, Hg, Al, Ga, In, Ge, Sn, Pb, a lanthanide, or SiR₂ where R is a C₁-C₈ saturated or unsaturated alkyl group.

122. A compound as claimed in claim 121, wherein M is Mg, Ca, Ti, V, Nb, Cr, Mo, Mn, Tc, Fe, Ru, Co, Rh, Ni, Pd, Pt, Cu, Ag, Au, Zn, Cd, Hg, Al, Ga, In, Ge, Sn, Pb or a lanthanide.

123. A compound as claimed in claim 122, wherein M is Zn, Cu, Cd, Ca, Mn, Au or Co.

124. A compound as claimed in claim 115 or claim 123, wherein M is Zn, Cd, Ca, Mn, Au or Co.

125. A compound as claimed in claim 124, wherein M is Zn.

126. A compound as claimed in any one of claims 113 to 125, wherein each R¹, R², R³, R⁴, R⁵, R⁶, R⁷, R⁸, R⁹, R¹⁰, R¹¹, R¹², R¹³ and R¹⁴ is independently hydrogen, methyl, ethyl, propyl, allyl, CO₂H, CH₂CO₂H or (CH₂)₂CO₂H.

127. A compound as claimed in any one of claims 113 to 126, wherein R¹ and R³ are hydrogen.

128. A compound as claimed in any one of claims 113 to 127, wherein R⁵, R⁸ and R¹¹ are hydrogen.

129. A compound as claimed in any one of claims 113 to 128, wherein R's is hydrogen, sodium, a C₁-C₆ saturated or unsaturated alkyl group or a naturally occurring amino acid.

130. A compound as claimed in any one of claims 113 to 129, wherein R¹⁵ is aspartic acid or lysine.

131. A compound as claimed in any one of claims 113 to 130, wherein the compound is substantially enantiomerically pure.

132. A compound as claimed in any one of claims 113 to 131, wherein R¹ and R³ are hydrogen, and R¹ is in the down-configuration and R³ is in the up-configuration in formula 3 as shown.

133. A compound as claimed in any one of claims 113 to 132, wherein R¹ and R³ are hydrogen, R² is (CH₂)₂CO₂H, R⁴ is CO₂H, and chiral centres 1* and 2* are in the (S)-configuration.

134. A compound as claimed in any one of claims 113 to 133, wherein the compound is of formula 2

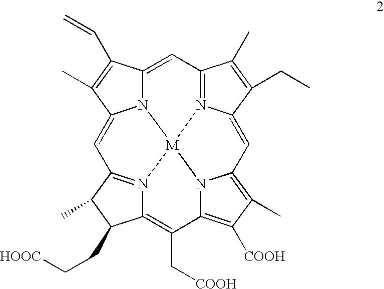

135. A method of carrying out an MRI scan, the method comprising using a compound as claimed in any one of claims 113 to 134 as an MRI enhancer.

136. Use of a compound as claimed in any one of claims 113 to 134 as an MRI enhancer.
